(19) United States
(12) Patent Application Publication
(10) Pub. No.: US 2024/0425797 A1
(43) Pub. Date: Dec. 26, 2024

(54) METHODS FOR DETECTING MICROORGANISMS

(71) Applicant: DIAMIDEX, MARSEILLE CEDEX 09 (FR)

(72) Inventors: SAM DUKAN, MARSEILLE (FR); AUDREY DUMONT, AUBAGNE (FR); EDITH GRAC, MARSEILLE (FR); ALEXANDRE MULLER, MARSEILLE (FR)

(21) Appl. No.: 18/701,274

(22) PCT Filed: Oct. 17, 2022
(86) PCT No.: PCT/EP2022/078875

§ 371 Date: Apr. 15, 2024

(30) Foreign Application Priority Data
Oct. 15, 2021 (EP) 21306445.4

(51) Int. Cl. C12M 1/34; G01N 15/01; G01N 15/06

(52) U.S. Cl. CPC C12M 41/36; G01N 15/01; G01N 15/0625

(57) ABSTRACT

The present invention relates to a method, and related device, for observing, and where appropriate detecting and optionally enumerating, microbiological objects on a support, such as a membrane or a solid growth medium. In particular, said method comprises a) illuminating an area of a support with at least two incident collimated light sources forming an angle (α) of at least 10° with respect to the normal of the support, b) acquiring an image of said area of said support illuminated by said at least two incident collimated light sources by means of a light receiving element having its optical acquisition axis along the normal of the support, and c) detecting the presence or the absence of microbiological objects on said area of said support by discrimination, on said acquired image, of light reflected, scattered and/or diffused from the support and from microbiological objects on said support.

### FIELD OF THE INVENTION

The present invention relates to the field of microbiology and more specifically to a method for observing, and where appropriate detecting and optionally enumerating, microbiological objects on a support, such as a membrane or a solid growth medium.

### BACKGROUND OF THE INVENTION

Microorganisms needs to be monitored in environment (bathing or drinking water) or in raw materials and final products designed for human and animal consumption or use such as food, beverages, medical devices, pharmaceutical or cosmetic products. The presence of microorganisms can cause not only the deterioration or spoilage of the product, but also disease if consumed by or administered to a human or animal. To ensure safety of these products, microbiological tests are required to examine the risk of contamination under normal use conditions and thereby preventing poisoning or infection outbreaks. In particular, for medical devices, pharmaceutical or cosmetic industry, need of sterility is very important and testing the microbiological quality of products and raw materials is important along the whole supply chain as possible flaws of products can occur at every stage of production.

Of course, microorganisms may also infect humans or animals and detecting and/or quantifying microorganisms responsible of an infection is required in clinical diagnosis. For instance, diagnosis of urinary tract infection (UTI) may performed through quantitative urine culture. Traditionally, the presence of 1,000 or more bacteria/ml in urine has been considered to represent significant bacteriuria, indicating UTI.

For all these industries, clinical applications and environmental monitoring, the contamination thresholds and the methods used may vary. However, revealing the presence microorganisms at the earliest possible stage is always crucial.

Culture on a Petri dish, the Louis Pasteur's method, has been the most effective way of identifying and counting microorganisms since the 19th century. To date, this technique still remains the reference method in many industries to detect and count microorganisms.

The method is simple: extracting the microorganisms from a sample, putting them on a dedicated Petri dish containing a suitable medium and counting colonies when they get visible to the eye. Though reliable, this method is long (up to several days according to the target microorganism).

In order to shorten the length of the assay, it was suggested to use indicators such as chromogenic substrates, fluorogenic substrates, or fluorescent or radio-labeled antibodies to detect smaller size microcolonies (e.g. International patent applications WO 96/14431, WO 2013/050598, WO 2008/118400). However, for most of these methods, the growth and indicator stages have to be segregated for optimal results: the growth stage for fast cell growth without harmful indicators therein that retard growth, and the indicator stage for dedicated coloring and identification that effectively allow smaller size microcolonies to be detected.

Over the years, many other attempts have been made to reduce the time required for the assay, in particular using culture independent approaches based on immunological methods, nucleic acid amplification methods, or flow cytometry methods using labeling by fluorescent antibodies or fluorogenic substrates. However, these methods remain expensive, have a not easy workflow, require highly skilled specialists and/or concentrated samples and, for most of them, it is very difficult to distinguish live and dead microorganisms.

Thus, there remains a need for a rapid, sensitive, user friendly and cost-effective method for detecting and/or enumerating microorganisms in a sample.

### SUMMARY OF THE INVENTION

The inventors herein provide a method that fulfils this need. More particular, they provide a new method allowing observation, and where appropriate detection, of microorganism colonies, at a very early stage in their growth, i.e. when they are of a diameter that is too small to be visible to the naked eye. This method does not require the use of any additional reagents facilitating the detection such as labelling agents.

In a first aspect, the present invention relates to a method for detecting microbiological objects on a support comprising
a) illuminating an area the support with at least two incident collimated light sources forming an angle (α) of at least 10° with respect to the normal of the support, the value of angle (α) being independently selected for each incident collimated light source,
b) acquiring an image of said area of said support illuminated by said at least two incident collimated light sources by means of a light receiving element having its optical acquisition axis along the normal of the support, and
c) detecting the presence or the absence of microbiological objects on said area of said support by discrimination, on said acquired image, of light reflected, scattered and/or diffused from the support and from microbiological objects on said support.

Preferably, the microbiological objects to be detected are selected from the group consisting of molds and colonies/microcolonies of bacteria, archaebacteria and yeasts, and combinations thereof, more preferably are selected from the group consisting of molds and colonies/microcolonies of bacteria and yeasts, and combinations thereof and even more preferably comprise molds and colonies/microcolonies of bacteria and/or yeasts.

Preferably, the microbiological objects to be detected are individualizable or individualized microbiological objects.

Preferably, the microbiological objects to be detected are not labelled with a compound or moiety generating a photonic signal.

Preferably, steps b) and c) are carried out a plurality of times to image several areas of said support, preferably to image the entirety of the support.

The method may further comprise a step d) of combining acquired images of said areas so as to form a combined image.

The support may be a membrane filter, preferably a membrane filter made of mixed cellulose esters (MCE), polyvinylidene fluoride (PVDF), nitrocellulose, polytetrafluoroethylene, polycarbonate or nylon, or combinations thereof, more preferably made of mixed cellulose esters (MCE), polyvinylidene fluoride (PVDF), polyester sulfone (PES), nitrocellulose, polytetrafluoroethylene, polycarbonate or nylon, even more preferably made of mixed cellulose esters (MCE) or polyvinylidene fluoride (PVDF).

Alternatively, the support may be a solid growth medium.

In some embodiments, the support is in a container and covered by a translucent lid.

In some embodiments, the method further comprises, after step b) and before step c),
b′) repeating steps a) and b) one or several times, each time with a different set of at least two incident collimated light sources forming an angle (α) of at least 10° with respect to the normal of the support, the value of angle (α) being independently selected for each incident collimated light source, thereby obtaining a plurality of acquired images of said area and combining said acquired images of said area so as to form a combined image of said area.

Preferably, in this embodiment, in step c) the presence or the absence of microbiological objects on said area of said support is detected by discrimination, on said combined image of said area, of light reflected, scattered and/or diffused from the support and from microbiological objects on said support.

In some embodiments, steps a) and b) or steps a), b) and b′) are carried out a plurality of times to image several areas of said support, preferably to image the entirety of the support. The method may further comprise after step b) or b′), b″) combining acquired images of said areas so as to form a combined image of said areas. In step c), the presence or the absence of microbiological objects on said areas of said support may be detected by discrimination, on said combined image of said areas, of light reflected, scattered and/or diffused from the support and from microbiological objects on said support.

Preferably, steps a) and b) are repeated 1, 2, 3, 4, 5, 6, 7, 8, 9 or 10 times with 1, 2, 3, 4, 5, 6, 7, 8, 9 or 10 different sets of at least two incident collimated light sources, respectively. More preferably, steps a) and b) are repeated 1, 2, 3, 4, 5, 6, 7 or 8 times with 1, 2, 3, 4, 5, 6, 7 or 8 different sets of at least two incident collimated light sources, respectively.

Preferably, the incident collimated light sources of two sets differ in their number, their type of light, their position and/or the value of angle α of each light sources. More preferably, the incident collimated light sources of two sets differ in their number, their position and/or the value of angle α of each light sources.

Preferably, the light receiving element is a camera comprising an array of pixel sensors, preferably a CCD (charge-coupled device) image sensor or an active pixel sensor such as CMOS (complementary metal-oxide-semiconductor) image sensor.

Preferably, in step a) (first time or repetition of step a), the support is illuminated with a set of at least three, preferably 3 to 24, more preferably 3 to 12, and even more preferably 3 to 6, incident collimated light sources. In particular, in step a) (first time or repetition of step a), the support may be illuminated with a set of three incident collimated light sources.

Preferably, said incident collimated light sources are evenly distributed on a circle centered on the optical acquisition axis. In particular, said incident collimated light sources of a set may be evenly distributed on one or several circles centered on the optical acquisition axis.

Each incident collimated light source forms an angle (α) which may be independently selected between 15° to 75° with respect to the normal of the support, preferably between 25° to 65° with respect to the normal of the support, more preferably between 30° to 60° with respect to the normal of the support.

Preferably, a set comprises at least three incident collimated light sources and the value of angle α is identical for all light sources of the set.

Preferably, said incident collimated light sources are selected from collimated light-emitting diodes (LED) and laser diodes, and combination thereof. More preferably said incident collimated light sources are collimated white LED.

The method may further comprise enumerating and/or identifying microbiological objects on the support from acquired image(s) or combined image.

The method may further comprise before step a) providing a sample to be tested, contacting said sample with the support and incubating said support in order to allow growth of microorganisms, if any.

The method of the invention may be used to detect microorganisms in a sample or to test the sterility of a sample.

In particular, the sample may be obtained from a liquid, a solid, or a gas. Preferably, the sample is selected from the group consisting of biological samples, environmental samples, medical devices or any part thereof, foods or beverages for human or animal consumption, pharmaceutical or cosmetic products, and ingredients of such foods, beverages, pharmaceutical or cosmetic products.

In a second aspect, the present invention relates to a device for detecting microbiological objects on a support, preferably a membrane filter or a solid growth medium, said device comprising
at least two incident collimated light sources for illuminating the support, said at least two incident collimated light sources forming an angle (α) of at least 10° with respect to the normal of the support, the value of angle (α) being independently selected for each incident collimated light source,
a light receiving element for acquiring an image of an area of said support illuminated by said at least two incident collimated light sources, said light receiving element having its optical acquisition axis along the normal of the support, and
means detecting the presence or the absence of microbiological objects on said area of said support by discrimination, on said acquired image, of light reflected, scattered and/or diffused from the support and from microbiological objects on said support, and optionally means to hold, and optionally move, the support.

Preferably, the device comprises at least 3, preferably 3 to 50, more preferably 3 to 24, and even more preferably 3 to 12, incident collimated light sources. Alternatively, the device may comprise at least 13 incident collimated light sources, preferably 15 to 50, more preferably 15 to 30, and even more preferably 24 incident collimated light sources.

Preferably, each incident collimated light source forms an angle (α) which is independently selected between 15° to 75°, preferably between 25° to 65°, more preferably between 30° to 60, with respect to the normal of the support.

Preferably, the value of angle α is identical for incident collimated light sources which are symmetrical with respect to the optical acquisition axis.

In particular, the device may comprise
(i) at least three, preferably 3, 4, 5, 6, 7, 8, 9, 10, 11 or 12, incident collimated light sources with a value of angle (α) between 45° and 55°, or
(ii) at least three, preferably 3, 4, 5 or 6, incident collimated light sources with a value of angle (α) between 30° and 40° and at least three, preferably 3, 4, 5 or 6, incident collimated light sources with a value of angle (α) between 45° and 55°, or
(iii) at least three, preferably 3, 4, 5 or 6, incident collimated light sources with a value of angle (α) between 25° and 35°, at least three, preferably 3, 4, 5 or 6, incident collimated light sources with a value of angle (α) between 40° and 50° and at least 6, preferably 7, 8, 9, 10, 11 or 12, incident collimated light sources with a value of angle (α) between 60° and 70°, or
(iv) at least three, preferably 3, incident collimated light sources with a value of angle (α) between 28° and 32°, at least three, preferably 3, incident collimated light sources with a value of angle (α) between 33° and 37°, at least three, preferably 3, incident collimated light sources with a value of angle (α) between 38° and 42°, at least three, preferably 3, incident collimated light sources with a value of angle (α) between 48° and 52°, at least three, preferably 3, incident collimated light sources s with a value of angle (α) between 53° and 57°, and at least three, preferably 3, 4, 5, 6, 7, 8, 9, incident collimated light sources with a value of angle (α) between 60° and 64°.

Preferably, the device comprises at least three incident collimated light sources, more preferably from 4 to 12 incident collimated light sources, said incident light sources being evenly distributed on a circle centered on the optical acquisition axis and wherein each incident light source forms an angle (α) which is independently selected between 15° to 75° with respect to the normal of the support, preferably between 30° to 60° with respect to the normal of the support.

The present invention also relates to the use of the device of the invention to detect, and optionally enumerate and/or identify, microbiological objects on a support, preferably according to the method of the invention.

The present invention also relates to the use of the device of the invention to detect microorganisms in a sample or to test the sterility of a sample, preferably using the method of the invention.

### BRIEF DESCRIPTION OF THE FIGURES

FIG. 1: Schematic view of an embodiment of the device of the invention.

FIG. 2: illustration of the technical result of the invention, applied to the detection of bacteria, yeasts and molds. Bacterial microcolony sizing about 200-500 μm diameter (P. aeruginosa, E. coli, R. piketti), 500-700 m (L. pneumophila, B. cepacia), or 1-2 mm (B. subtilis). Yeast cells sizing about 500 μm diameter (yeast C. albicans), or molds fitting in ˜5×5 mm window size (A. brasiliensis). Each image indicates scale factor with white line representing about 100 μm. Microorganisms were grown on PDVF membrane, black mixed ester cellulose (MCE) membrane, white MCE membrane or black polyethersulfone (PES) membrane.

FIG. 3: illustration of the technical result of the invention, applied to the detection of bacteria, yeasts and molds. The incidence angle α was set to 37°, 50°, or 60°. Each image indicates scale factor with white line representing about 100 μm. Microorganisms were grown on PDVF membrane, black MCE membrane or white MCE membrane.

FIG. 4: illustration of the technical result of the invention, applied to the detection of bacteria, yeasts and molds. The membrane was illuminated with white light, UV light, blue light or red light. Among the possible reflected lights, only the one carrying the same colour as illumination was collected. Microorganisms were grown on PDVF membrane or black PES membrane. Each image indicates scale factor with white line representing about 100 μm.

FIG. 5: illustration of the technical result of the invention, applied to the detection of bacteria, yeasts and molds. In reference condition “no lid”, the membrane was not covered and was directly exposed to incidence light. In other conditions, a lid was added on top of the membrane (between the membrane and the light illumination/receiving light system). The distance between the lid and the membrane was set to 1 to 3 mm. In these conditions, incident light had to go through the lid before reaching the sample, and reflected light had to go through the lid before reaching the sensor. Three types of material for the lid were investigated: Lid 1: plastic from Merck filtration units (Merck Millipore, EZ-FIT Filtration units Ref EFHVW10IS), Lid 2:UV grade fused silica (Chroma corp., thickness: 1 mm), Lid 3=sodalime glass (Selba corp., thickness: 1.8-2 mm). Microorganisms were grown on PDVF membrane or white MCE membrane. Each image indicates scale factor with white line representing about 100 μm.

FIG. 6: illustration of the technical result of the invention, applied to the detection of bacteria, yeasts and molds. Microorganisms were grown on PDVF, black MCE, white MCE, black PES or nylon membrane. Each image indicates scale factor with white line representing about 100 μm.

FIG. 7: illustration of the technical result of the invention, applied to the detection of A. brasiliensis or E. coli. Microorganisms were detected either directly on the surface of the solid growth medium or on a PVDF or MCE membrane deposited on the surface of the solid growth medium.

FIG. 8: Technical result of the invention, applied to the detection of C. alibcans. Microorganisms were grown on white MCE membrane, either grinded or ungrided, detected after 23, 25, or 28 hours growth on the solid growth medium. For each timing investigated, the counting of the number of C. albicans detected was carried out in standard condition 0 (3 leds, incidence angle α set to 50°), condition 1 (6 leds, incidence angle α set to 37°), or condition 2 (12 leds, 6 of them with incidence angle α set to 50°, 6 of them with α=37°), the result is represented in percentage of detection by MICA vs the counting on the same membrane after reincubation for 48 h again until colonies became visible by naked eye.

### DETAILED DESCRIPTION OF THE INVENTION

The object of the present invention is to provide a method and the corresponding device for detecting, and optionally enumerating, microbiological objects, e.g. microorganisms or microorganism colonies, present on a support, at a very early stage in their growth. The inventors herein demonstrated that this method provides a very flexible solution to detect a great variety of microorganisms, on several types of supports, and in various conditions, including in closed containers to prevent contamination through the environment.

This method further exhibits the advantage of allowing early detection of these microbiological objects without requiring, while allowing, the use of any additional reagents facilitating the detection such as labelling agents or dyes.

According, in a first aspect, the present invention relates to a method for detecting microbiological objects on a support comprising
a) illuminating an area of the support with at least two incident collimated light sources forming an angle (α) of at least 10° with respect to the normal of the support,
b) acquiring an image of said area of said support illuminated by said at least two incident collimated light sources by means of a light receiving element having its optical acquisition axis along the normal of the support, and
c) detecting the presence or the absence of microbiological objects on said area of said support by discrimination, on said acquired image, of light reflected, scattered and/or diffused from the support and from microbiological objects on said support.

As used herein, the term “microbiological object” refers to a microorganism in an isolated form (e.g. a mold) or a cluster of microorganisms, i.e. a colony or microcolony of microorganisms. Preferably, the microbiological object has a dimension (e.g. diameter, length or width) of at least 10 μm. More preferably, this microbiological object has a dimension (e.g. diameter, length or width) of at least 10 μm and is invisible or barely visible with the naked eye. The microbiological objects to be detected by the method of the invention may comprise, or consists of, microorganisms in an isolated form, in particular molds, colonies or microcolonies, and mixtures thereof, i.e. a combination of (i) microorganisms in an isolated form and (ii) colonies or microcolonies.

The microbiological objects to be detected by the method of the invention are preferably individualizable or individualized microbiological objects. In particular, these objects are preferably not comprised in a microbial lawn. An individualizable microbiological object is an object whose contours can be visually distinguished from contours of other microbiological objects. An individualizable microbiological object can be isolated from other objects or can be partially overlapped by other objects, e.g. two overlapping colonies. An individualized microbiological object is an object that is isolated from other objects, e.g. an isolated colony or microcolony or an isolated mold. In preferred embodiments, the microbiological objects to be detected by the method of the invention are individualizable or individualized (i) molds and/or (ii) colonies/microcolonies of bacteria, archaebacteria and/or yeasts. In particular, the microbiological objects to be detected are selected from the group consisting of molds and colonies/microcolonies of bacteria, archaebacteria and yeasts, and combinations thereof, preferably are selected from the group consisting of molds and colonies/microcolonies of bacteria and yeasts, and combinations thereof. In particular, the microbiological objects to be detected may comprise molds and colonies/microcolonies of bacteria and/or yeasts.

In some preferred embodiments, the microbiological objects comprises, or consists of, microcolonies. As used herein, the term “microcolonies” refers to colonies which have grown for several hours or days depending of the microorganism and which are invisible or barely visible with the naked eye. Typically, the diameter of microcolonies is less than 500 μm, preferably between 10 μm and 500 μm, more preferably between 30 am and 500 μm, even more preferably between 30 am and 200 μm.

As used herein, the term “microorganism” refers to a bacterium, an archaebacterium or a microscopic fungus. Preferably, this term refers to a bacterium, a yeast (i.e. unicellular microscopic fungus) or a mold (i.e. multicellular and filamentous microscopic fungus). The microorganisms to be detected may be selected from the group consisting of bacteria, archaebacteria and microscopic fungi, and combinations thereof. Preferably, the microorganisms to be detected are selected from the group consisting of bacteria and microscopic fungi, and combinations thereof. The microorganisms to be detected may be pathogenic or non-pathogenic microorganisms. Preferably, the microorganisms are pathogenic microorganisms or non-pathogenic, for instance microorganisms which can deteriorate or spoil a product (e.g. food, beverage, medical device, pharmaceutical or cosmetic product) or an environment (e.g. swimming-pool, groundwater).

In some embodiments, the microorganisms to be detected comprise bacteria. These bacteria can be detected in the form of colony or microcolony, preferably in the form of microcolony. These bacteria may be an aerobic, anaerobic or facultative anaerobic bacteria, and Gram negative or Gram positive bacteria. In particular, the microorganisms to be detected may comprise bacteria belonging to the genera Aeromonas (e.g. Aeromonas hydrophila), Alcaligenes (e.g. Alcaligenes faecalis), Acinetobacter (e.g Acinetobacter aceti, Acinetobacter baumanii), Alicyclobacillus (e.g. Alicyclobacillus acidiphilus, Alicyclobacillus acidocaldarius, Alicyclobacillus acidoterrestris, Alicyclobacillus contaminans, Alicyclobacillus cycloheptanicus, Alicyclobacillus herbarius, Alicyclobacillus hesperidum), Asaia (e.g. Asaia siamensis), Bacillus (e.g. Bacillus subtilis), Brevundimonas (e.g. Brevundimonas diminuta), Burkholderia (e.g. Burkholderia cepacia), Citrobacter (e.g. Citrobacter freundii), Clostridium (e.g. Clostridium sporogenes), Cutibacterium (e.g. Cutibacterium acnes), Edwardiella (e.g. Edwardiella tarda), Enterobacter (e.g. Enterobacter aerogenes), Enterococcus (e.g. Enterococcus faecalis), Escherichia (e.g. Escherichia coli), Gluconoacetobacter (e.g. Gluconoacetobacter liquefaciens), Gluconobacter (e.g. Gluconobacter oxydans), Klebsiella (e.g. Klebsiella pneumoniae), Lactobacillus (e.g. Lactobacillus casei, Lactobacillus nagelii, Lactobacillus plantarum), Legionella (e.g. Legionella pneumophila), Methylobacterium (e.g. Methylobacterium extorquens), Micrococcus (e.g. Micrococcus luteus), Moraxella (e.g. Moraxella osloensis), Ochrobactrum (e.g. Ochrobactrum anthropi), Pantoea (e.g. Pantoea agglomerans), Pediococcus (e.g. Pediococcus pentosaceus), Proteus (e.g. Proteus mirabilis), Pseudomonas (e.g. Pseudomonas aeruginosa), Ralstonia (e.g. Ralstonia pickettii), Salmonella (e.g. Salmonella typhimurium), Serratia (e.g. Serratia marcescens), Stenotrophomonas (e.g. Stenotrophomonas maltophilia), Shigella (e.g. Shigella sonnei), Sphingomonas (e.g. Sphingomonas paucimobilis), Staphylococcus (e.g. Staphylococcus aureus), Streptococcus (e.g. Streptococcus pneumoniae), Vibrio (e.g. Vibrio parahaemolyticus), Weissella (e.g. Weissella confusa), Yersinia (e.g. Yersinia enterolitica), and combinations thereof.

In some embodiments, the microorganisms to be detected comprise yeasts or molds. Yeasts can be detected in the form of colony or microcolony, preferably in the form of microcolony. Molds can be detected in an isolated form (detection of a unique organism). In particular, the microorganisms to be detected may comprise yeast or molds belonging to the genera Aspergillus (e.g. Aspergillus brasiliensis), Candida (e.g. Candida albicans), Geotrichum (e.g. Geotrichum candidum), Penicillium (e.g. Penicillium chrysogenum, Penicillium variotii), Saccharomyces (e.g. Saccharomyces cerevisiae), Zygosaccharomyces (e.g. Zygosaccharomyces bailii), and combinations thereof.

The microbiological objects to be detected may be contained in a sample to be analyzed. This sample may be obtained from a liquid (e.g. water, fruit juices, beer, wine, biological fluids such as urine), a solid (e.g. food, pharmaceutical or cosmetic products, medical devices or any solid surfaces) or a gas (e.g. air). Depending on the form of the product/environment to be tested, the sample may be directly allowed to grow on the support or may be submitted to preliminary step(s) before to be allowed to grow on the support.

In particular, the sample can be a liquid sample or a liquified sample. As used herein, the term “liquified sample” refers to a liquid sample obtained from a solid sample. In some cases, the solid sample may be dissolved or suspended in a liquid medium through physical and/or chemical treatments. Microorganisms may also be extracted from different surfaces or devices using any method known by the skilled person such as swabbing methods, friction methods, e.g. using wipes, printing methods, e.g. by agar contact method, rinsing or immersion methods, or sonication methods, in particular to dislodge biofilms (see e.g. Ismail et al., Int J Environ Res Public Health. 2013 Nov. 14; 10(11):6169-83, incorporated herein by reference).

Liquid samples may contain suspended solids. However, if necessary, remaining suspended solids may be eliminated from the liquid medium using a suitable method, preferably using a method minimizing the loss of microorganisms, e.g. by low speed centrifugation or filtration using suitable pore size. The liquid medium used to suspend solid sample may be any suitable solvent such as sterile water, buffer solution or liquid culture medium.

Optionally, the liquid or liquified sample may be diluted (e.g. serial dilutions) or concentrated prior step a) using any suitable method such as centrifugation or filtration.

The sample analyzed with the method of the invention can be any sample for which it is sought to determine whether it is contaminated with microorganisms.

Examples of samples include, but are not limited to, biological samples (e.g. saliva, nasopharyngeal, urine, fecal, blood, plasma, cerebrospinal fluid or mucus sample), environmental sample (e.g. residential, commercial or industrial water, waste water, cooling water, boiler water, ground water, recreational water, process water, effluent of water treatment unit, soil, or other environmental material), medical devices or any part thereof, foods or beverages for human or animal consumption (e.g. dairy products, raw materials, drinking water, fruit juices, beers, wines, water used in the composition of the product), pharmaceutical or cosmetic products, as well as ingredients of such foods, beverages, pharmaceutical or cosmetic products.

Before being observed with the method of the invention, the microbiological objects are allowed to grow on the support. In particular, the sample may be contacted with the support in order to allow growth of microorganisms if any. The support may be a membrane filter or a solid growth medium.

As used herein, the term “growth medium” or “culture medium” refers to a nutrient medium used for growth of microorganisms within the context of the present invention. The growth medium can be a defined medium synthesized from individual chemicals so the exact molecular composition is known, or an undefined medium comprising some complex ingredients, such as yeast extract or casein hydrolysate, which consist of a mixture of many chemical species in unknown proportions. The growth medium used in the method of the invention may be a non-selective growth medium or a selective growth medium. A non-selective growth medium is a general media for bacterial growth, for fungi growth or for both bacterial and fungi growth. A non-selective medium typically contains the nutrients required to support the growth of a wide variety of microorganisms. A selective growth medium is a medium used for the growth of only selected microorganisms. Indeed, the composition of a selective medium ensures the proliferation of cells with certain properties, such as antibiotic resistance or the ability to synthesize a certain metabolite, e.g. an amino acid. This medium thus provides an environment that favors the growth of the target microorganisms over nontarget microorganisms that may be present in a sample.

The expression “solid growth medium” or “solid culture medium” as used herein refers to a growth medium which allows microorganisms to form microcolonies on its surface, such as a medium which has a gel-like appearance or is in the form of a gel, a gel being a colloidal system in which a porous network of interconnected particles spans the volume of a liquid medium and allows nutrients to diffuse through the medium to become available to the microorganisms. Preferably, the solid growth medium as used herein is prepared by adding to a liquid growth medium a sufficient amount of a gelling agent such as agar, agarose, alginate, carrageenan, cellulose, gelatin, pectin and combinations thereof. Typically, the solid growth medium contains a gelling agent, preferably agar, at a concentration of 0.5% to 3%, preferably 1% to 2.5%. Preferably, the solid growth medium used in the method of the invention is an agar growth medium. The solid growth medium is poured in a container. This contained can be any sterile container adapted to microbial culture, in particular a conventional Petri plate.

In embodiments wherein the support is a solid growth medium, the method may further comprise, before step a), contacting a sample, in a container, with a solid growth medium containing nutrients to support growth of the microorganisms of interest.

In embodiments wherein the support is a membrane filter, the method may further comprise, before step a), concentrating the microorganisms of a sample on a membrane filter and then contacting said membrane with a growth medium, e.g. a solid growth medium or a pad soaked with a liquid growth medium, preferably a solid growth medium. The passage of nutrients through the filter during incubation allows the growth of microorganisms on the upper surface of the membrane.

The liquid, liquified or gaseous sample may be filtered/concentrated using a sterile membrane filter suitable to retain microorganisms contained in the sample, typically a microfiltration membrane filter having pore sizes smaller than the target microorganisms. The sample may be passed through the membrane using a filter funnel and vacuum system.

Preferably, the membrane filter has a nominal pore size not greater than 1.2 μm, in particular a pore size of 0.22 μm to 1.2 μm or a pore size of 0.22 μm to 0.8 μm. In some preferred embodiments, the membrane filter has a nominal pore size not greater than 0.45 μm, in particular a pore size of 0.22 μm to 0.45 μm. The diameter of the filter may depend on the device used to filtrate the sample and the size of the container containing the growth medium. For example, the container may be a 100 mm, 90 mm or 55 mm diameter Petri dish and the membrane may have a diameter of 47 mm, 50 mm or less.

The membrane filter may be made of any suitable material such as mixed cellulose esters (MCE), polyvinylidene fluoride (PVDF), polyester sulfone (PES), nitrocellulose, polytetrafluoroethylene, polycarbonate or nylon, or a combination thereof. Preferably, the membrane filter is made of mixed cellulose esters (MCE), polyvinylidene fluoride (PVDF), nitrocellulose, polytetrafluoroethylene, polycarbonate or nylon. More preferably the membrane filter is made of mixed cellulose esters (MCE) or polyvinylidene fluoride (PVDF). The membrane filter may be white or colored, e.g. black, and/or may be grid membrane.

In preferred embodiments, sample dilution and/or filtered sample volume is/are adjusted in order to get a maximum of 1000, preferably a maximum of 300, microbiological objects/cm² of membrane filter or solid growth medium surface.

In embodiments wherein the support is a membrane filter, said membrane may be kept on the growth medium during the steps of illumination and image acquisition (steps a) and b), optionally repeated) or may be removed from said growth medium and placed on a sample holder before step a). Optionally, the membrane may be placed on the sample holder in a container, e.g. a petri dish. Said container may be an open container or may be closed with a translucent lid.

In embodiments wherein the support is a solid growth medium, said support is in a container, e.g. a petri dish. Before step a) of the method, said container may be opened by removing the lid used during the culture, or may be kept closed, i.e. with a translucent lid.

In these embodiments, the translucent lid is placed opposite the microbiological objects, between the microbiological objects and the light receiving element, and is not in contact with said objects. Preferably, the translucent lid is placed at a distance of the microbiological objects which is between 1 mm and 10 cm, more preferably between 1 mm and 5 cm, even more preferably between 1 mm and 1 cm.

In step a) of the method of the invention, an area of the support is illuminated with a set of at least two incident collimated light sources forming an angle (α) of at least 10° with respect to the normal of the support.

Incident light sources are electric powered light sources. They may illuminate the support with any pattern such as a pattern being a point, a line, a circle or a square.

As used herein, the term “collimated light source” refers to a light source generating parallel rays of light. A collimated light therefore spreads minimally as it propagates. The terms “collimated light source”, “incident collimated light source”, “incident light source” and “light source” are interchangeably used in the present document. Collimated incident light sources are preferably selected from collimated light-emitting diodes (LED) and laser diodes, and combination thereof. More preferably, collimated incident light sources are collimated LED.

The light sources used in the present invention may emit monochromatic, polychromatic, or even white light. In particular, the light sources may emit visible light (i.e. having wavelengths in the range of 400 to 700 nm), infrared light (i.e. having wavelengths higher than 700 nm) or ultraviolet light (i.e. having wavelengths lower than 400 nm). Preferably, the light sources emit light having wavelengths in the range of 320 to 750 nm, more preferably in the range of 400 nm to 700 nm.

In an embodiment, each light source of the set emits a light independently selected from the group consisting of white light, UV light (preferably having wavelengths in the range of 320 to 400 nm), green light (preferably having wavelengths in the range of 530 to 600 nm), blue light (preferably having wavelengths in the range of 430 nm to 530 nm) and red light (preferably having wavelengths in the range of 600 nm to 700 nm).

In a preferred embodiment, each light source of the set emits a light independently selected from the group consisting of white light, UV light (preferably having wavelengths in the range of 320 to 400 nm), blue light (preferably having wavelengths in the range of 430 nm to 530-nm) and red light (preferably having wavelengths in the range of 600 nm to 700 nm).

The light sources of the set may emit the same type of light or different type of lights. As illustration, each light source of the set may provide white light. Alternatively, at least one light source of the set may provide white light and at least one other light source of the set may provide UV light. Preferably, each light source of the set emits the same type of light, preferably emits white light.

In a particular embodiment, the light sources of the set are selected from the group consisting of collimated white LED and collimated LED emitting UV, blue or red light, and combinations thereof. Preferably, the light sources of the set are collimated white LED.

In a preferred embodiment, all light sources of the set are selected from the group consisting of collimated white LED and collimated LED emitting UV, blue or red light, and combinations thereof. Preferably, all light sources of the set are collimated white LED.

In preferred embodiments, the incident light of the set is not filtered through an excitation filter, i.e. a filter commonly used in fluorescence microscopy and spectroscopic applications for selection of the excitation wavelength of light from a light source, before reaching the microbiological objects or the support.

As mentioned above, the method of the invention does not require the use of any additional reagents facilitating the detection such as labelling agents or dyes. However, in some particular embodiments, microbiological objects may have been labelled using such reagents, e.g. fluorescent dyes. Preferably, the microbiological objects are unlabeled, i.e. are not labelled with a labelling agent, i.e. a compound or moiety generating a photonic signal such as a colored or fluorescent dye. In preferred embodiments, the method of the invention does not encompass the use of any reagent (e.g. substrate, probe, antibody, dye, etc.) to label microbiological objects.

In step a) the support is illuminated with a set of at least two light sources, preferably with a set of at least 3 light sources. In particular, the support may be illuminated with a set of 3 to 50 light sources, preferably with a set of 3 to 24 light sources, more preferably a set of with 3 to 12 light sources, and even more preferably with a set of 3 to 6 light sources.

In preferred embodiments, the support is illuminated with a set of 3 light sources.

Preferably, the light sources of the set are distributed, preferably evenly distributed (i.e. equidistant from each other), on one or several circles centered on the optical acquisition axis. In some embodiments, said light sources are distributed, preferably evenly distributed, on a circle centered on the optical acquisition axis. In some other embodiments, said light sources are distributed, preferably evenly distributed, on several circles, preferably of different diameters, centered on the optical acquisition axis, preferably on 2, 3, 4, 5, or 6 circles. Preferably, the circle(s) is(are) in a plane parallel to the plane of the support.

In an embodiment, the support is illuminated with a set of 3 light sources evenly distributed on a circle centered on the optical acquisition axis.

In some particular embodiments, the support is illuminated with an even number of light sources and said light sources are symmetrical with respect to the optical acquisition axis.

The value of angle α is independently selected for each light source of the set. Thus, the value of angle α of two light sources of the set may be identical or different. Preferably, each incident collimated light source of the set forms an angle (α) which is independently selected between 15° and 75° with respect to the normal of the support, preferably between 25° and 65° with respect to the normal of the support, more preferably between 30° and 60° with respect to the normal of the support.

In a particular embodiment, the value of angle α is identical for light sources of the set which are symmetrical with respect to the optical acquisition axis.

In a preferred embodiment, the value of angle α is identical for all light sources of the set.

In a particularly preferred embodiment, the support is illuminated with a set of at least three light sources and the value of angle α is identical for all light sources. In particular, in this embodiment, the value of angle α may be between 25° and 65° with respect to the normal of the support.

The luminous flux emitted by each light source and the sum of luminous flux emitted by all light sources may be easily adjusted by the skilled person. Preferably, the sum of luminous flux emitted by all light sources of a set is between 0.80 to 15 lumen/mm² and the luminous flux emitted by each light source is between 0.10 and 5 lumen/mm².

The area of the support illuminated by the collimated light sources may be a portion of the support or the entirety of the support. Preferably, the area of the support illuminated by the collimated light sources is a portion of the support. In particular, said area may be from 3 to 100 mm², preferably from 3 to 50 mm², more preferably of 25 to 50 mm². Typically, the entire area of the support is comprised between 100 mm² and 10,000 mm², preferably between 400 mm² and 8,000 mm², more preferably between 450 and 2000 mm².

The distance between the support and the light receiving element and light sources may be easily adjusted by the skilled person, by the operator or automatically, e.g. using a computer image analysis software. In particular, this distance may be determined using a triangulation method. Typically, the distance between the support and the light receiving element is from 5 mm to 10 cm, preferably from 5 mm to 5 cm, and more preferably from 5 mm to 3 cm.

In step b) of the method of the invention, an image of said area of said support illuminated by said at least two incident collimated light sources is acquired by means of a light receiving element having its optical acquisition axis along the normal of the support.

The light receiving element may be a camera comprising an array of pixel sensors, preferably a CCD (charge-coupled device) image sensor or an active pixel sensor such as CMOS (complementary metal-oxide-semiconductor) image sensor. Preferably, the light receiving element is a CMOS image sensor.

One of the advantages of the present invention is to detect microbiological objects at a very early stage of growth without the need of magnification. Thus, preferably, the light receiving element is a non-magnifying system. However, the use of such system is not excluded, and images may be acquired for example at 0.5× to 10× magnification, preferably at 2× to 5× magnification.

In some embodiments, the method of the invention further comprises, after step b) and before step c),
b′) repeating steps a) and b) one or several times, each time with a different set of light sources, thereby obtaining a plurality of acquired images of said area and combining said acquired images of said area so as to form a combined image of said area.

Steps a) and b) may be repeated 1 to 20 times with 1 to 20 different sets of at least two incident collimated light sources. In particular, steps a) and b) may be repeated 1, 2, 3, 4, 5, 6, 7, 8, 9 or 10 times with 1, 2, 3, 4, 5, 6, 7, 8, 9 or 10 different sets of at least two incident collimated light sources, respectively. Preferably, steps a) and b) are repeated 1, 2, 3, 4, 5, 6, 7 or 8 times with 1, 2, 3, 4, 5, 6, 7 or 8 different sets of at least two incident collimated light sources, respectively. More particularly, steps a) and b) are repeated 1, 2, 3, 4 times with 1, 2, 3 or 4 different sets of at least two incident collimated light sources, respectively.

Each set of light sources may be as defined above. All embodiments disclosed above for the set used in step a) are also considered for each set of light sources used in repeated steps. In particular, each set may comprise at least 2, preferably at least 3, light sources forming an angle (α) of at least 10° with respect to the normal of the support, the value of angle (α) being independently selected for each light source. Preferably, the value of angle α is identical for each light source of the same set.

Each set may comprise 3 to 24, preferably 3 to 12, more preferably, 3 to 6, and even more preferably 3, light sources. The number of light sources may be identical or different for each set. In particular, all set may comprise a different number of light sources, all set may comprise the same number of light sources, or some of the sets may comprise the same number of light sources.

Preferably, each set comprises at least three light sources and the value of angle α is identical for all light sources of a set. Preferably, the value of angle α is between 250 and 650 with respect to the normal of the support.

Each set of light sources comprises a different combination of light sources and differs from the other set(s) in at least one light source, preferably differs from all light sources.

The light sources of two sets may differ in their number, their type of light, their position and/or the value of angle α of each light sources. Preferably, the light sources of two sets differ in their number, their position and/or the value of angle α of each light sources. More preferably, the light sources of two sets differ at least in their position and/or the value of angle α of each light sources.

Preferably, the light sources of all sets emit the same type of light preferably white light. More preferably, the light sources of all sets are collimated white LED.

An image of the area is acquired for each set of light sources. Said images are then combined in order to obtain a combined image of said area.

It should be noted that the method of the invention does not exclude the possibility of repeating steps a) and b) one or several times with the same set of light sources.

In particular, the method of the invention may comprise
a) illuminating an area of the support with a set of at least two, preferably at least three, incident collimated light sources forming an angle (α) of at least 10° with respect to the normal of the support, the value of angle (α) being independently selected for each incident collimated light source,
b) acquiring an image of said area of said support illuminated by said set of at least two, preferably at least three, incident collimated light sources by means of a light receiving element having its optical acquisition axis along the normal of the support, and
b′) repeating steps a) and b) one or several times, each time with a different set of at least two, preferably at least three, incident collimated light sources forming an angle (α) of at least 10° with respect to the normal of the support, the value of angle (α) being independently selected for each incident collimated light source, thereby obtaining a plurality of acquired images of said area and combining said acquired images of said area so as to form a combined image of said area.

In a particular embodiment, the method of the invention may comprise
a) illuminating an area of the support with a first set of at least two, preferably at least three, incident collimated light sources forming an angle (α) of at least 10° with respect to the normal of the support, the value of angle (α) being independently selected for each incident collimated light source,
b) acquiring an image of said area of said support illuminated by said first set of at least two, preferably at least three, incident collimated light sources by means of a light receiving element having its optical acquisition axis along the normal of the support, and
b′) repeating steps a) and b) with a second set of at least two, preferably at least three, incident collimated light sources forming an angle (α) of at least 10° with respect to the normal of the support, the value of angle (α) being independently selected for each incident collimated light source, thereby obtaining a plurality of acquired images of said area and combining acquired images of said area so as to form a combined image of said area.

Preferably, the value of angle (α) of each incident collimated light source of the first set is identical. More preferably, the value of angle (α) of each incident collimated light source of the first set is identical and is between 25° and 65°.

Preferably, the value of angle (α) of each incident collimated light source of the second set is identical. More preferably, the value of angle (α) of each incident collimated light source of the second set is identical and is between 25° and 65°.

The value of angle (α) of each incident collimated light source of the first set and the value of angle (α) of each incident collimated light source of the second set may be identical or different. In a particular embodiment, the value of angle (α) of each incident collimated light source of the first set and the value of angle (α) of each incident collimated light source of the second set are identical, preferably are between 25° and 55°, more preferably between 30° and 40°. In another particular embodiment, the value of angle (α) of each incident collimated light source of the first set and the value of angle (α) of each incident collimated light source of the second set are different, preferably the value of angle (α) of each incident collimated light source of the first set is between 30° and 40° and the value of angle (α) of each incident collimated light source of the second set is between 45° and 55°, or vice-versa.

In a particular embodiment, the method of the invention may comprise
a) illuminating an area of the support with a first set of at least two, preferably at least three, incident collimated light sources forming an angle (α) of at least 10° with respect to the normal of the support, the value of angle (α) being independently selected for each incident collimated light source,
b) acquiring an image of said area of said support illuminated by said first set of at least two, preferably at least three, incident collimated light sources by means of a light receiving element having its optical acquisition axis along the normal of the support, and
b′) repeating steps a) and b) with each of N sets of at least two, preferably at least three, incident collimated light sources, each light sources of each set forming an angle (α) of at least 10° with respect to the normal of the support, the value of angle (α) being independently selected for each incident collimated light source, thereby obtaining a plurality of acquired images of said area and combining acquired images of said area so as to form a combined image of said area.

N is an integer and is between 2 and 20, preferably between 2 and 12, more preferably between 2 and 8, i.e. is selected from the group consisting of 2, 3, 4, 5, 6, 7 and 8.

Preferably, the value of angle (α) of each incident collimated light source of the first set is identical. More preferably, the value of angle (α) of each incident collimated light source of the first set is identical and is between 25° and 65°.

For each of the N sets, the value of angle (α) of each incident collimated light source of the same set may be identical or different. Preferably, the value of angle (α) of each incident collimated light source of the same set is identical. More preferably, the value of angle (α) of each incident collimated light source of the same set is identical and is between 25° and 65°.

In a particular embodiment, the value of angle (α) of each incident collimated light source of the same set is identical, preferably between 25° and 65°, and the value of angle (α) is different for each of the N sets.

In another particular embodiment, the value of angle (α) of each incident collimated light source of the same set is identical, preferably between 25° and 65°, and two or more sets have the same value of angle (α).

In another particular embodiment, the value of angle (α) of each incident collimated light source of the same set is identical, four sets of light sources are used, two sets with a value of angle (α) between 30° and 40° and two sets with a value of angle (α) between 45° and 55°. Preferably, each set comprises at least three light sources, preferably 3 to 6 light sources, more preferably consists of three light sources.

In another particular embodiment, the value of angle (α) of each incident collimated light source of the same set is identical, at least 8 sets of light sources are used, two sets with a value of angle (α) between 25° and 35° two sets with a value of angle (α) between 40° and 50° and four sets with a value of angle (α) between 60° and 70°. Preferably, each set comprises at least three light sources, preferably 3 to 6 light sources, more preferably consists of three light sources.

In another particular embodiment, the value of angle (α) of each incident collimated light source of the same set is identical, at least 8 sets of light sources are used, one set with a value of angle (α) between 28° and 32°, one set with a value of angle (α) between 33° and 37°, one set with a value of angle (α) between 38° and 42°, one set with a value of angle (α) between 48° and 52°, one set with a value of angle (α) between 53° and 57°, and three sets with a value of angle (α) between 60° and 64°. Preferably, each set comprises at least three light sources, preferably consists of three light sources. Preferably, each set comprises at least three light sources, preferably 3 to 6 light sources, more preferably consists of three light sources.

In embodiments comprising steps a), b) and b′), in step c) the presence or the absence of microbiological objects on said area of said support may be detected by discrimination, on said combined image of said area, of light reflected, scattered and/or diffused from the support and from microbiological objects on said support.

In some preferred embodiments, steps a) and b) (with one set of light sources) or steps a), b) and b′) (repetition with a plurality of light source sets) are carried out a plurality of times to image several areas of said support, preferably to image the entirety of the support. Thus, the method may further comprise, after step b) or b′), b″) combining acquired images of said areas so as to form a combined image of said areas. In step c) the presence or the absence of microbiological objects on said areas of said support may be then detected by discrimination, on said combined image of said areas, of light reflected, scattered and/or diffused from the support and from microbiological objects on said support.

Steps a) and b) or steps a) to c) may be carried out a plurality of times to image several different areas of said support. Preferably, steps a) and b) or steps a) to c) are carried out to image the entirety of the support, i.e. are carried out a number of times sufficient to image the entirety of the support by combining acquired images.

In particular, the steps of illumination and image acquisition of an area (steps a) and b)), and optionally combination of acquired images of said area when steps a) and b) are repeated with one or several different sets of light sources, may be carried out a plurality of times to image several different areas of said support, preferably to image the entirety of the support by combining acquired images of different areas.

The number of rounds depends on the size of the area imaged at each round and the size of the support. Typically, steps a) and b) or steps a) to c) may be carried out from 10 times to 500 times, preferably from 50 times to 400 times.

In particular, the steps of illumination and image acquisition of an area (steps a) and b)), and optionally combination of acquired images of said area when steps a) and b) are repeated with one or several different sets of light sources, may be carried out from 10 times to 500 times, preferably from 50 times to 400 times.

The method may further comprise a step of combining acquired images of said different areas so as to form a combined image of the support. For each illuminated area, an image may be acquired, said area images then being processed so as to form a combined image of the support. Alternatively, for each illuminated area, several images may be acquired and combined, said area combined images then being processed so as to form a combined image of the support. The combined image then constitutes a representation of the support, in its entirety or a portion thereof. In preferred embodiments, step c) is conducted on such combined image of the support, in its entirety or a portion thereof.

The manipulation of images such as combination of acquired images of the same area to form a combined image of an area or combination of acquired or combined images of different areas to form a combined image of the support, can be done by any method routinely used by the skilled person. For example, combination of images can be done using ImageJ software.

The support is advantageously mounted on a moving sample holder, which makes it possible to scan the surface of the support and to reconstitute a partial or complete image of said support. The width of an incident area (illuminated and imaged area) is preferably equal or bigger to the translation step between two successive positions of the support. Thanks to the moving sample holder, the support may be scan by a linear and/or rotational movement of the sample relative to the light receiving element.

The detection of the presence or the absence of microbiological objects on the support is carried out by discrimination on the acquired image(s) or combined image, of light reflected, scattered and/or diffused from the support and from microbiological objects on said support. A microbiological object adds certain irregularities on the surface of the support, and this can be detected thank to the difference of light reflection, scattering and/or diffusion on the support and on said microbiological object. The light reflected, scattered and/or diffused from the support and from microbiological objects on said support is distinct from a photonic signal emitted by a labelling agent, in particular from a fluorescent signal emitted by a fluorescent dye, or an auto-fluorescent signal emitted by the microbiological objects. In particular, the light reflected, scattered and/or diffused from the support and from microbiological objects on said support has the same wavelength range than the incident light. In a particular embodiment, the incident collimated light sources emit white light and the light receiving element detect white light reflected, scattered and/or diffused from the support and from microbiological objects on said support. The same is true for other types of lights (e.g. blue, red, green UV lights).

This detection can be carried out by an operator or else automatically by means of a computer executing image processing software connected to the light receiving element. Such computer executing image processing software may be any suitable software easily chosen by the skilled person.

In particular, the detection of microbiological objects may be carried out by detecting reflection spots arising from surface irregularities given by microbiological objects. For example, this spot detection may be carried out (i) by using the number of pixels receiving a signal level above a threshold value around a light intensity peak for each line or column of an array of pixels of the pixel sensor, (ii) by using the highest signal level of each line or column of an array of pixels of the pixel sensor, or (iii) by using the signal level of the array of pixel sensor.

In some embodiments, the method of the invention further comprises before step a) providing a sample to be tested, contacting said sample with the support and incubating said support in order to allow growth of microorganisms, if any. The sample, the support and the incubation conditions may be as defined above.

This detection can be accompanied by counting the microbiological objects, and also by their classification/identification according to a given criterion, for example their surface area or their morphology.

The method of the invention can thus further comprise enumerating and/or identifying microbiological objects on the support from acquired image(s) or combined image.

As used herein, the term “identification” does not necessarily require the determination of genus and species of a given microbiological object. This term may refer to a classification of a microorganism in a taxonomic group (at any rank) or in a specific group (e.g. thermophilic acidophilic bacteria, acetic acid bacteria, lactic acid bacteria, yeasts or molds). This identification may be possible via information obtained from acquired image(s) or combined image (size, morphology, aspect) and/or merely via the detection of said object, in particular if the culture step has been performed on a selective growth medium and/or under selective conditions (e.g thermophilic conditions).

In some preferred embodiments, the method of the invention is used to test the sterility of a sample or to detect microorganisms in a sample. In particular, before step a), the sample may be contacted with the support and incubated in order to allow growth of microorganisms as detailed above. The presence of a microbiological object or the presence of a number of microbiological objects which is higher than a predetermined threshold indicating that the sample is not sterile. The absence of microbiological object or the presence of a number of microbiological objects which is lower than a predetermined threshold indicating that the sample is sterile. This threshold may vary according to the product to be tested and the regulation standard to be applied.

The invention method of the invention allows to detect the microbiological objects with high reliability after an incubation time of typically 4 days or less, preferably 3, 2, 1 day or less, depending on the nature of the microbiological objects to be detected.

In another aspect, the present invention relates to a device for performing the method of the invention, especially for detecting microbiological objects on a support, preferably a membrane filter or a solid growth medium, said device comprising
at least two incident collimated light sources for illuminating the support, said at least two incident collimated light sources forming an angle (α) of at least 10° with respect to the normal of the support,
a light receiving element for acquiring an image of an area of said support illuminated by said at least two incident collimated light sources, said light receiving element having its optical acquisition axis along the normal of the support, and
means for detecting the presence or the absence of microbiological objects on said area of said support by discrimination, on said acquired image, of light reflected, scattered and/or diffused from the support and from microbiological objects on said support, and
optionally means to hold, and optionally move, the support.

Incident light sources may be any collimated light sources as defined above. In particular, incident light sources are electric powered light sources. They may illuminate the support with any pattern such as a pattern being a point, a line, a circle or a square.

Light sources are preferably selected from collimated light-emitting diodes (LED) and laser diodes, and combination thereof. More preferably, collimated incident light sources are collimated LED.

The light sources used in the present invention may emit monochromatic, polychromatic, or even white light. In particular, the light sources may emit visible light (i.e. having wavelengths in the range of 400 to 700 nm), infrared light (i.e. having wavelengths higher than 700 nm) or ultraviolet light (i.e. having wavelengths lower than 400 nm). Preferably, the light sources emit light having wavelengths in the range of 320 to 750 nm, more preferably in the range of 400 nm to 700 nm.

In an embodiment, each light source emits a light independently selected from the group consisting of white light, UV light (preferably having wavelengths in the range of 320 to 400 nm), green light (preferably having wavelengths in the range of 530 to 600 nm), blue light (preferably having wavelengths in the range of 430 nm to 530-nm) and red light (preferably having wavelengths in the range of 600 nm to 700 nm).

In a preferred embodiment, each light source emits a light independently selected from the group consisting of white light, UV light (preferably having wavelengths in the range of 320 to 400 nm), blue light (preferably having wavelengths in the range of 430 nm to 530 nm) and red light (preferably having wavelengths in the range of 600 nm to 700 nm).

The light sources may emit the same type of light or different type of lights. As illustration, each light source may provide white light. Alternatively, at least one light source may provide white light and at least one other light source may provide UV light. Preferably, each light source emits the same type of light, preferably emits white light.

In a particular embodiment, said at least two incident collimated light sources are selected from the group consisting of collimated white LED and collimated LED emitting UV, blue or red light, and combinations thereof. Preferably, said at least two incident collimated light sources are collimated white LED.

In a preferred embodiment, all light sources are selected from the group consisting of collimated white LED and collimated LED emitting UV, blue or red light, and combinations thereof. Preferably, all light sources are collimated white LED.

The device may comprise at least 3 incident collimated light sources, preferably 3 to 50, more preferably 3 to 24, and even more preferably 3 to 12 incident collimated light sources. In preferred embodiment, the device may comprise at least 4 incident collimated light sources, preferably 4 to 50, more preferably 4 to 24, and even more preferably 4 to 12 incident collimated light sources. In another preferred embodiment, the device comprises at least 13 incident collimated light sources, preferably 15 to 50, more preferably 15 to 30, and even more preferably 24 incident collimated light sources.

Preferably, said light sources are distributed, preferably evenly distributed (i.e. equidistant from each other), on one or several circles centered on the optical acquisition axis. In some embodiments, said light sources are distributed, preferably evenly distributed, on a circle centered on the optical acquisition axis. In some other embodiments, said light sources are distributed, preferably evenly distributed, on several circles, preferably of different diameters, centered on the optical acquisition axis, preferably on 2, 3, 4, 5, or 6 circles. Each circle is in a plane parallel to the plane of the support. Preferably, each circle comprises the positions of at least three light sources, preferably the positions of 3 to 12 light sources, more preferably, the positions of 3 to 9 light sources. In preferred embodiments, each light source positioned on the same circle has the same value of angle α.

In some particular embodiments, the device comprises an even number of light sources and said light sources are symmetrical with respect to the optical acquisition axis.

The value of angle α is independently selected for each light source. Thus, the value of angle α of two light sources of the device may be identical or different. Preferably, each incident collimated light source forms an angle (α) which is independently selected between 15° to 75° with respect to the normal of the support, preferably between 25° to 65°, more preferably between 30° to 60° with respect to the normal of the support.

In a particular embodiment, the value of angle α is identical for light sources which are symmetrical with respect to the optical acquisition axis.

In a particular embodiment, the value of angle α is identical for all light sources.

In another particular embodiment, the device comprises at least three, preferably 3, 4, 5, 6, 7, 8, 9, 10, 11 or 12, light sources with a value of angle (α) between 45° and 55°. Preferably, the device comprises 12 incident collimated light sources with a value of angle (α) between 45° and 55°. Said light sources may be distributed, preferably evenly distributed on one or several circles centered on the optical acquisition axis. Preferably, said light sources are distributed, preferably evenly distributed, on one circle centered on the optical acquisition axis.

In another particular embodiment, the device comprises at least three, preferably 3, 4, 5 or 6, light sources with a value of angle (α) between 30° and 40° and at least three, preferably 3, 4, 5 or 6, light sources with a value of angle (α) between 45° and 55°. Preferably, the device comprises 6 light sources with a value of angle (α) between 30° and 40° and 6 light sources with a value of angle (α) between 45° and 55°. Said light sources may be distributed, preferably evenly distributed on one or several circles centered on the optical acquisition axis. Preferably, said light sources are distributed, preferably evenly distributed, on two circles centered on the optical acquisition axis. In particular, a first circle may comprise the positions of light sources with a value of angle (α) between 30° and 40° and a second circle may comprise the positions of light sources with a value of angle (α) between 45° and 55°.

In another particular embodiment, the device comprises at least three, preferably 3, 4, 5 or 6, light sources with a value of angle (α) between 25° and 35°, at least three, preferably 3, 4, 5 or 6, light sources with a value of angle (α) between 40° and 50° and at least 6, preferably 7, 8, 9, 10, 11 or 12, light sources with a value of angle (α) between 60° and 70°. Preferably, the device comprises 6 light sources with a value of angle (α) between 25° and 35°, 6 light sources with a value of angle (α) between 40° and 50° and 12 light sources with a value of angle (α) between 60° and 70°. Said light sources may be distributed, preferably evenly distributed on one or several circles centered on the optical acquisition axis. Preferably, said light sources are distributed, preferably evenly distributed, on three circles centered on the optical acquisition axis. In particular, a first circle may comprise the positions of light sources with a value of angle (α) between 25° and 35°, a second circle may comprise the positions of light sources with a value of angle (α) between 40° and 50° and the third circle may comprise the positions of light sources with a value of angle (α) between 60° and 70°.

In another particular embodiment, the device comprises at least three, preferably 3, light sources with a value of angle (α) between 28° and 32°, at least three, preferably 3, light sources with a value of angle (α) between 33° and 37°, at least three, preferably 3, light sources with a value of angle (α) between 38° and 42°, at least three, preferably 3, light sources with a value of angle (α) between 48° and 52°, at least three, preferably 3, light sources with a value of angle (α) between 53° and 57°, and at least three, preferably 3, 4, 5, 6, 7, 8, 9, light sources with a value of angle (α) between 60° and 64°. Preferably, the device comprises 3 light sources with a value of angle (α) between 28° and 32°, 3 light sources with a value of angle (α) between 33° and 37°, 3 light sources with a value of angle (α) between 38° and 42°, 3 light sources with a value of angle (α) between 48° and 52°, 3 light sources with a value of angle (α) between 53° and 57°, and 9 light sources with a value of angle (α) between 60° and 64. Said light sources may be distributed, preferably evenly distributed on one or several circles centered on the optical acquisition axis. Preferably, said light sources are distributed, preferably evenly distributed, on six circles centered on the optical acquisition axis. In particular, a first circle may comprise the positions of light sources with a value of angle (α) between 28° and 32°, a second circle may comprise the positions of light sources with a value of angle (α) between 33° and 37°, a third circle may comprise the positions of light sources with a value of angle (α) between 38° and 42°, a 4th circle may comprise the positions of light sources with a value of angle (α) between 48° and 52°, a 5th circle may comprise the positions of light sources with a value of angle (α) between 53° and 57°, and a 6th circle may comprise the positions of light sources with a value of angle (α) between 60° and 64°.

The luminous flux emitted by each light source and the sum of luminous flux emitted by all light sources may be easily adjusted by the skilled person. Preferably, the sum of luminous flux emitted by all light sources is between 0.80 to 15 lumen/mm² and the luminous flux emitted by each light source is between 0.10 and 5 lumen/mm².

In preferred embodiments, the device does not comprise any excitation filter, i.e. a filter for selection of the excitation wavelength of light from incident light sources.

The light receiving element is as defined above. In particular, the light receiving element may be a camera comprising an array of pixel sensors, preferably a CCD (charge-coupled device) image sensor or an active pixel sensor such as CMOS (complementary metal-oxide-semiconductor) image sensor. Preferably, the light receiving element is a CMOS image sensor.

The light receiving element may be a magnifying system or a non-magnifying system. Magnification may be comprised between 0.5× and 10×, preferably between 2× and 5×. Preferably, the light receiving element is a non-magnifying system.

The light sources and the light receiving element are mounted together on a supporting structure so as to have a defined and fixed positional relationship with respect to each other.

The device comprises means for detecting the presence or the absence of microbiological objects on the support.

The device preferably comprises means for combining images, in particular combining acquired images of the same area and combining acquired or combined images of the several areas. The detection of the presence or the absence of microbiological objects on the support can thus be carried out on an acquired image of an area of the support, on a combined image of an area of the support (combination of several acquired images of the same areas) or on a combined image of several areas of the support.

The light receiving element may be connected with a detecting unit which will receive the image information obtained from the light receiving element and process this information to detect any microbiological object, i.e. a computer executing image processing software known per se.

Therefore, by detecting the light reflected, scattered and/or diffused from the support and from microbiological objects on said support, it is possible to discriminate light reflected, scattered and/or diffused from the support and from microbiological objects on said support and thus to detect the presence or absence of microbiological object on said support. A microbiological object adds certain irregularities on the surface of the support and this can be detected thank to the difference of light reflection, scattering and/or diffusion on the support and on said microbiological object. Indeed, irregularities given by microbiological objects have a specific structure, giving specific reflection pattern.

The light reflected, scattered and/or diffused from the support and from microbiological objects on said support is distinct from a photonic signal emitted by a labelling agent, in particular from a fluorescent signal emitted by a fluorescent dye, or an auto-fluorescent signal emitted by the microbiological objects. In particular, the light reflected, scattered and/or diffused from the support and from microbiological objects on said support has the same wavelength range than the incident light. In a particular embodiment, the incident collimated light sources emit white light and the light receiving element detect white light reflected, scattered and/or diffused from the support and from microbiological objects on said support. The same is true for other types of lights (e.g. blue, red, green UV lights).

In particular, the detection of microbiological objects may be carried out by detecting reflection spots arising from surface irregularities given by microbiological objects. For example, this spot detection may be carried out (i) by using the number of pixels receiving a signal level above a threshold value around a light intensity peak for each line or column of an array of pixels of the pixel sensor, (ii) by using the highest signal level of each line or column of an array of pixels of the pixel sensor, or (iii) by using the signal level of the array of pixel sensor.

Optionally, the device may further comprise means to hold, and optionally move, the support. Preferably, the device comprises a moving sample holder which makes it possible to scan the surface of the support, as described above. Thanks to the moving sample holder, the support may be scan by a linear and/or rotational movement of the sample relative to the light receiving element.

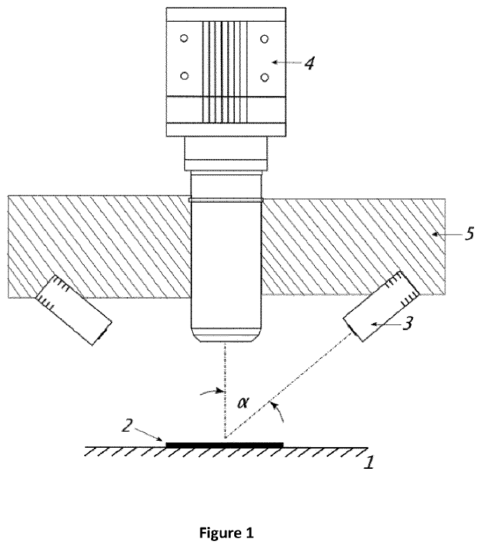

FIG. 1 shows an embodiment of the device of the invention. This exemplary device comprises two incident collimated light sources (3) for illuminating the support (2), said incident collimated light sources forming an angle (α) of at least 10° with respect to the normal of the support,
a light receiving element (4) for acquiring an image of an area of said support illuminated by said at least two incident collimated light sources, said light receiving element having its optical acquisition axis along the normal of the support,
means for detecting the presence or the absence of microbiological objects on said area of said support by discrimination, on said acquired image, of light reflected, scattered and/or diffused from the support and from microbiological objects on said support (not shown), and
a sample holder (1) to hold the support,
the light sources and the light receiving element being mounted together on a supporting structure (5) so as to have a defined and fixed positional relationship with respect to each other.

In a further aspect, the present invention also relates to the use of a device of the invention to detect, and optionally enumerate and/or identify, microbiological objects on a support, preferably according to the method of the invention.

In particular, the present invention relates to the use of a device of the invention to test the sterility of a sample or to detect microorganisms in a sample, preferably according to the method of the invention.

All embodiments described above for the method and the device of the invention are also contemplated in this aspect.

As used herein, a range defined by the term “between X and Y” encompasses the limits of this range, i.e. encompasses the values X and Y.

All the references cited in this description are incorporated by reference in the present application. Others features and advantages of the invention will become clearer in the following examples which are given for purposes of illustration and not by way of limitation.

### EXAMPLES

### Example 1

### Material and Methods

### Preparation of Microorganisms

For A. brasiliensis, BioBalls® from Biomérieux containing a precise number of microorganisms (BioBall® Multishot 550 Aspergillus brasiliensis SKU number: 56001) were diluted in NaCl 0.9% to obtain a defined number of microorganisms per experimental condition impacted on a filtration membrane (density ˜100 objects/membrane). The membrane was deposited on SDA agar plates (Pancreatic Digest of Casein 5 g/L, Peptic Digest of Animal Tissue 5 g/L, Dextrose 40 g/L, Agar 15 g/L, pH value 5.6±0.2) and micro-organisms were allowed to grow for 17 h or 18 h at 32.5° C. Alternatively, the membrane was deposited on R2A agar plates (casein acid hydrolysate 0.5 g/L, dextrose 0.5 g/L, dipotassium phosphate 0.3 g/L, magnesium sulfate 0.024 g/L, proteose peptone 0.5 g/L, sodium pyruvate 0.3 g/L, starch, soluble 0.5 g/L, yeast extract 0.5 g/L, agar 15 g/L, pH value 7.2±0.2) and micro-organisms were allowed to grow 38 h or 44 h at 22.5° C.

Candida albicans (ATCC® 10231™), Pseudomonas aeruginosa (ATCC® 10145™) Escherichia coli (ATCC® 8739™), Bacillus subtilis (ATCC® 6633™), Burkholderia cepacia (ATCC® 25608™), Ralstonia pickettii (ATCC® 27511™) and Legionella pneumophila (CIP 105349) were diluted in NaCl 0.9% from cryopreserved stocks previously quantified to obtain a defined number of microorganisms per experimental condition. The resulting suspensions were filtered on membrane and micro-organisms were allowed to grow t hours at temperature T on solid growth media, i.e. on SDA, R2A, TSA (Tryptone 15 g/L, Soja papainic peptone 5 g/L, Sodium chloride 5 g/L, Agar 15 g/L, pH value 7.3±0.2) or GVPC (glycine vancomycim polymixin cyclohexamid, purchased from Oxoid, Catalog ref:PO5074A) agar plates.

Membranes used in the example were as follow: PVDF membrane (Durapore HVWG04700, Merck Millipore), MCE black membrane (HABG047, Merck Millipore), MCE white grinded membrane (HAWG047, Merck Millipore), MCE white ungrided membrane (GE 10401670), PES membrane (metricel 66585, Pall laboratory) and nylon membrane (nylaflo 66608, Pall laboratory).

TABLE 1
Conditions used to grow micro-organisms on filtration membrane.

| | | Incubation | Incubation |
|---|---|---|---|
| | Growth | temperature | time |
| Micro-organism | medium | T (° C.) | t (hr) |
| A. brasiliensis | SDA | 32.5° C. | 17 h, 18 h |
| | R2A | 22.5° C. | 38 h, 44 h |
| C. albicans | SDA | 32.5° C. | 17 h |
| | | 22.5° C. | 28 h |
| P. aeruginosa | TSA | 37° C. | 13 h, 15 h |
| | TSA | 32.5° C. | 17 h |
| E. coli | TSA | 32.5° C. | 8 h |
| B. subtilis | TSA | 32.5° C. | 14 h |
| B. cepacia | TSA | 32.5° C. | 17 h |
| | | | 19 h |
| R. pickettii | R2A | 32.5° C. | 24 h |
| L. pneumophilia | GVPC | 37° C. | 50 h |

For each sample, growth was stopped by removing the membrane from the solid growth medium and the sample was stored at 4° C. until imaging.

### Sample Preparation

The membranes were deposited on a sample holder (1) made of black anodised aluminium along with 3 drops of sterile water (about 50-2501l) to rehydrate and keep the membrane humidified. The cassette was then placed in position for imaging as illustrated in FIG. 1.

### Imaging System Setup

Membranes were imaged through a device as illustrated in FIG. 1 wherein the membrane (2) was placed on a sample holder (1) and was illuminated with incident collimated LED (3) forming an angle (α) with respect to the normal of the support. An incident area of the membrane was imaged by means of a CMOS image sensor (4) having its optical acquisition axis along the normal of the membrane. The light reflected/scattered and/or diffused from the membrane and/or the microbiological objects on the membrane was detected.

Acquisition parameters (light source intensities between 0.7 and 11 lumen/LED and camera exposure time between 100 and 300 ms) were adjusted to maximize the signal-to-noise ratio. A multi-band emission filter (435±20 nm, 546±10 nm, and 690±50 nm bandpass) was placed between the CMOS image sensor and the sample. The CMOS sensor used in the example was Sony IMX178LU-C (IDS imaging camera UI-3880CP-M-GL_R2 or UI-3880CP-C-GL_R2).

In standard configuration, the membrane was illuminated by 3 collimated LEDs emitting white light, equidistant from each other and centred on the optical acquisition axis. The incidence angle α was set to 50°.

In the examples described below, several parameters of this configuration were varied, i.e. the LED number, the value of angle α and the wavelength of incident light. Further parameters were investigated such as the membrane type and the presence or absence of a translucent lid on the membrane.

### Results

### Variation of LED Number

The membrane was illuminated by collimated LEDs emitting white light, equidistant from each other and centred on the optical acquisition axis. The incidence angle α was set to 50°. The number of LEDs was varied between 3, 6, or 12 and several types of membrane were tested (PVDF membrane, white and black MCE membranes, PES membrane). With increased LED number, the individual LED intensity was reduced to keep the overall intensity unchanged (the overall light reaching the membrane was kept within the same order of magnitude than the condition with 3 LEDs). For each of these conditions, acquisitions parameters (camera exposure time and light intensity) were adjusted manually to generate similar membrane background (gray level of the same order of magnitude, whatever the condition).

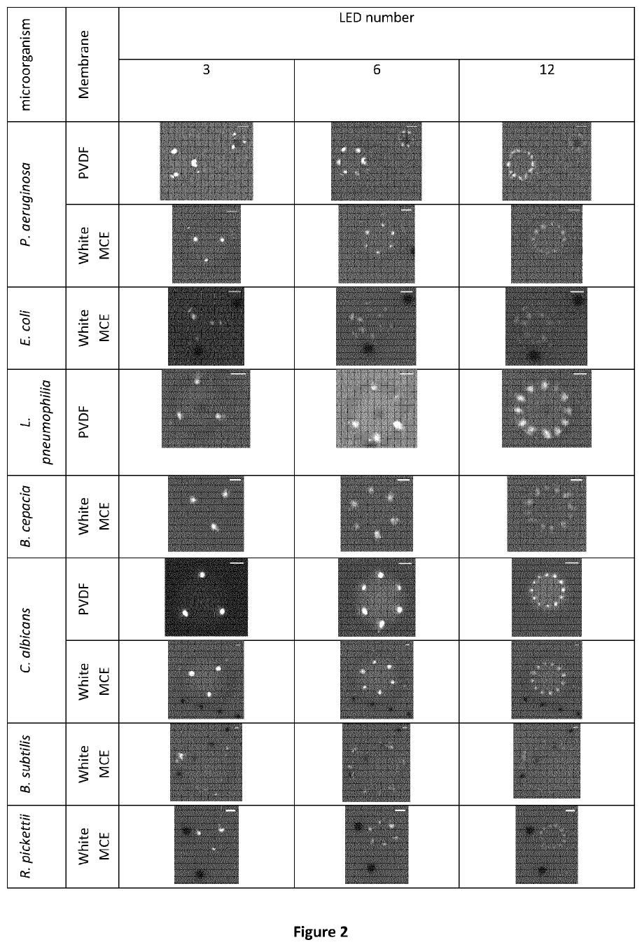

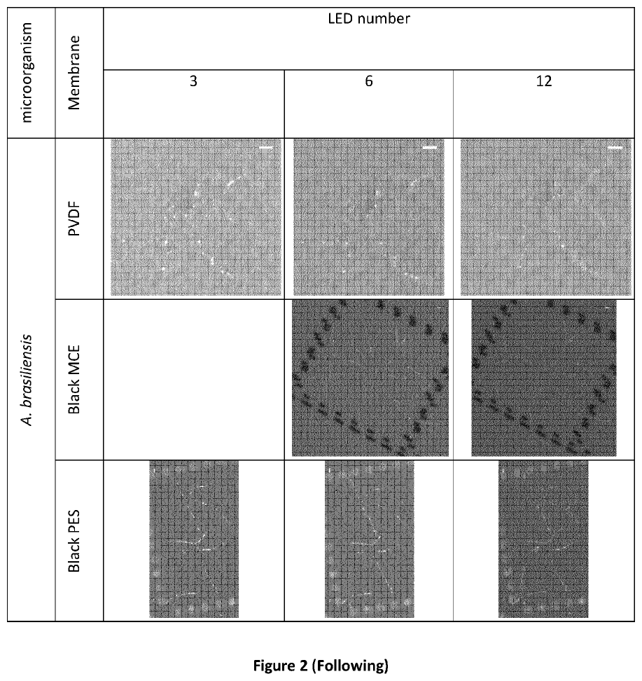

Examples of images acquired in each condition are presented in FIG. 2. These results showed that reflection spots are found on microorganisms, whatever the LED number. Those spots are well defined for microorganisms having a spherical-type-shape (such as C. albicans or P. aeruginosa). Unperfect spherical-type-shape can result in missing spots. However, the use of multiple light sources multiplies the information and thus allows efficient detection of each type of microorganisms.

### Variation of the Value of Angle α

The membrane was illuminated by 6 collimated LEDs emitting white light, equidistant from each other and centred on the optical acquisition axis. The incidence angle α was set to 37°, 50° or 60° and several types of membrane were tested (PVDF membrane, white and black MCE membranes).

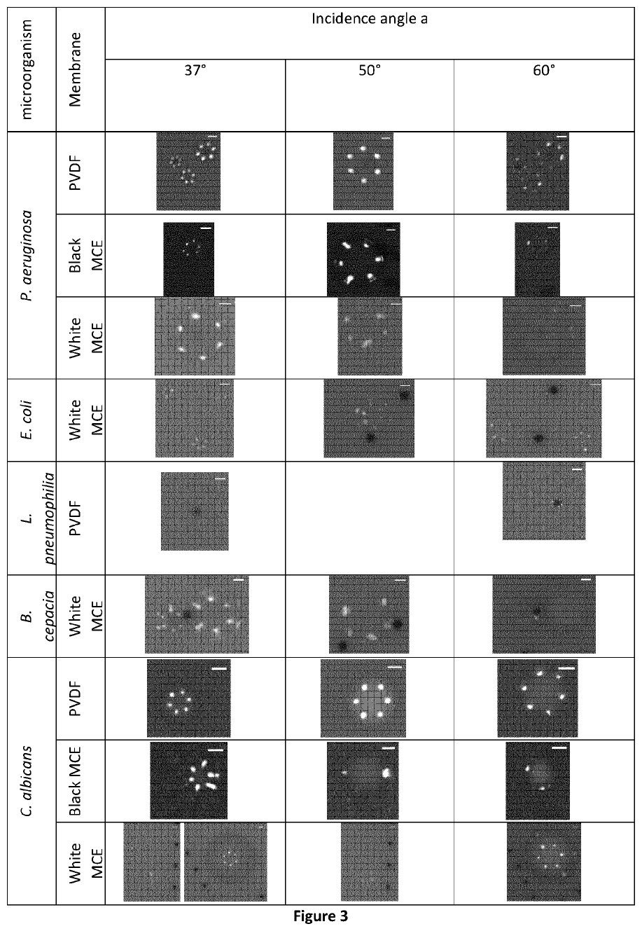

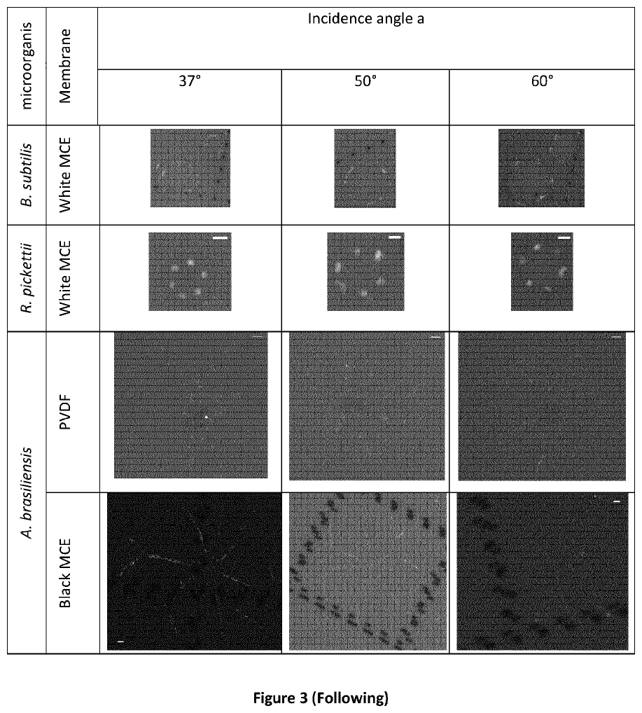

Examples of images acquired in each condition are presented in FIG. 3. These results showed that reflection spots are found on microorganisms, whatever the incidence angle. Small incidence angles (37°) provide reflection spots located near micro-organisms center. Indeed, flat surfaces (presumably found on microcolonies center) deviates only slightly the light from small incidence angle toward the sensor: the reflected light can be collected. By contrast, stiff surfaces (presumably found at microorganism edges) deviate light far away from the sensor: the reflected light cannot be collected.

On the other side, high incidence angles (e.g. 60°) lead to reflection spots located near the edges of micro-organisms. Indeed, flat surfaces (presumably found on microcolonies center) deviate light far away from the sensor: the reflected light cannot be collected. By contrast, stiff surfaces (presumably found at microorganism edges) deviates only slightly the light from small incidence angle toward the sensor: the reflected light can be collected.

These results show that the three angle values (37°, 50° and 60°) allow efficient detection of each type of microorganisms and, in some cases, it is possible to improve detection by adjusting the incidence angle according to the structure of the microorganisms to be detected or by using a combination of different incident angles.

### Variation of the Wavelength

The membrane was illuminated by 3 collimated LEDs equidistant from each other and centred on the optical acquisition axis. The incidence angle α was set to 50° and two types of membrane were tested (PVDF and PES membranes). The LEDs emitted white color, UV light (365±10 nm), blue light (495±20 nm) or red light (645±30 nm). In “white light” condition, the sensor collected the light within 435±20 nm, 546±10 nm, and 690±50 nm bandpass. In “UV light” condition, the sensor collected the light within 365±10 nm bandpass. In “Blue light” condition, the sensor collected the light within 482±35 nm bandpass. In “Red light” condition, the sensor collected the light within 640±75 nm bandpass.

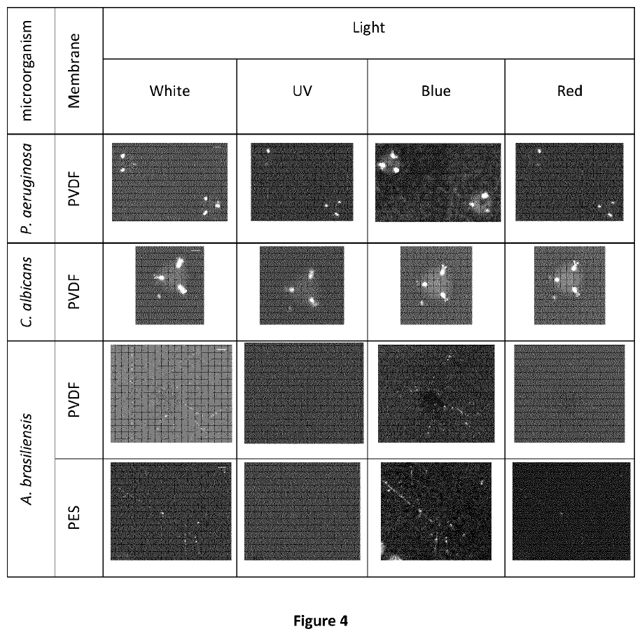

Examples of images acquired in each condition are presented in FIG. 4. These results show that each type of microorganisms can be efficiently detected whatever the light/wavelength.

### Presence of a Translucent Lid

The membrane covered by a translucent lid was illuminated by 12 collimated LEDs emitting white light, equidistant from each other and centred on the optical acquisition axis. The incidence angle α was set to 50° and two types of membrane were tested (PVDF and white MCE membranes). Three types of material for the lid were investigated: plastic from Merck filtration units, UV grade fused silica (Chroma corp., thickness:1 mm), sodalime glass (Selba corp., thickness:1.8-2 mm).

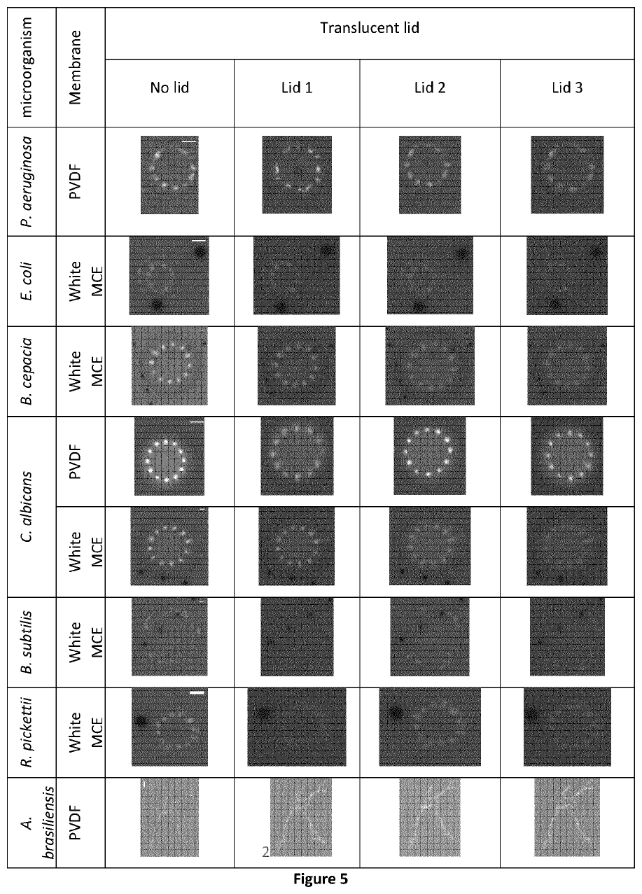

Examples of images acquired in each condition are presented in FIG. 5. These results show that each type of microorganisms can be efficiently detected even if the membrane is covered with a translucent lid and whatever the material of said lid.

### Membrane Type

The method of the invention was investigated for different kinds of membrane: PDVF, black MCE, white MCE, black PES or nylon membrane. For each condition, the membrane was illuminated by 6 collimated LEDs emitting white light, equidistant from each other and centred on the optical acquisition axis. The incidence angle α was set to 37°.

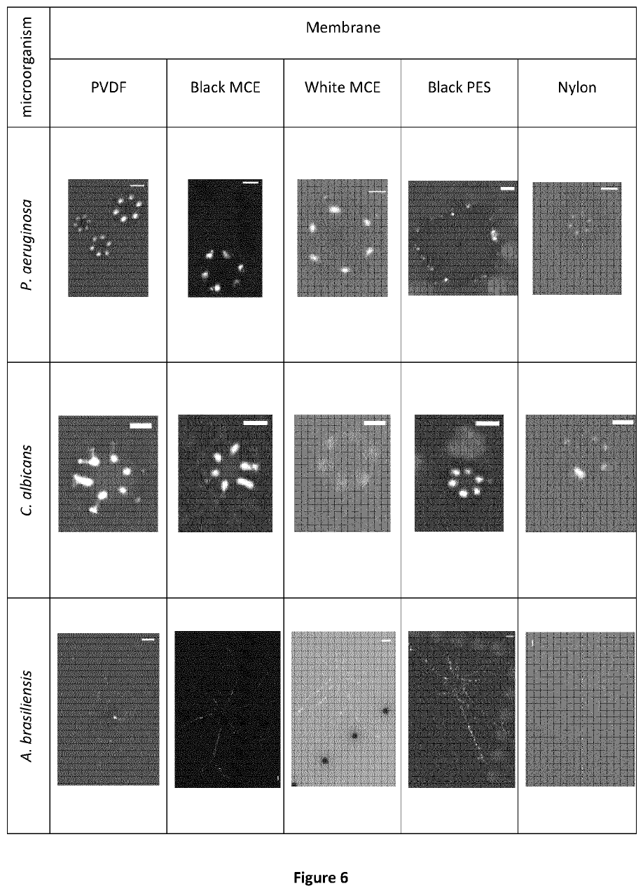

Examples of images acquired in each condition are presented in FIG. 6. These results show that each type of microorganisms can be efficiently detected on all tested membranes.

Detection on a Solid Growth Medium Escherichia coli (ATCC® 8739™) were diluted from glycerol stock. The resulting suspension was either spread on TSA agar plate (Tryptone 15 g/L, Soja papainic peptone 5 g/L, Sodium chloride 5 g/L, Agar 15 g/L, pH value 7.3±0.2), either filtered on a membrane (PVDF or White MCE). The membrane was deposited on TSA plate. In either case, bacteria were allowed to grow for 8 h at 37° C.

A. brasiliensis (BioBall® Multishot 550 Aspergillus brasiliensis SKU number: 56001 from Biomérieux containing a precise number of microorganisms) were diluted in NaCl 0.9% and were either spread on R2A plates (Pancreatic Digest of Casein 5 g/L, Peptic Digest of Animal Tissue 5 g/L, Dextrose 40 g/L, Agar 15 g/L, pH value 5.6±0.2), either impacted on a PVDF filtration membrane at a density ≈100 objects/membrane and deposited on R2A plates. In either case, micro-organisms were allowed to grow for 44 h at 22.5° C.

A. brasiliensis or E. coli microorganisms were then detected either directly on the surface of the solid growth medium or on the membrane deposited on the surface of the solid growth medium (90 mm diameter Petri-dish). In either case, the sample were deposited on a sample holder (1) made of black plastic.

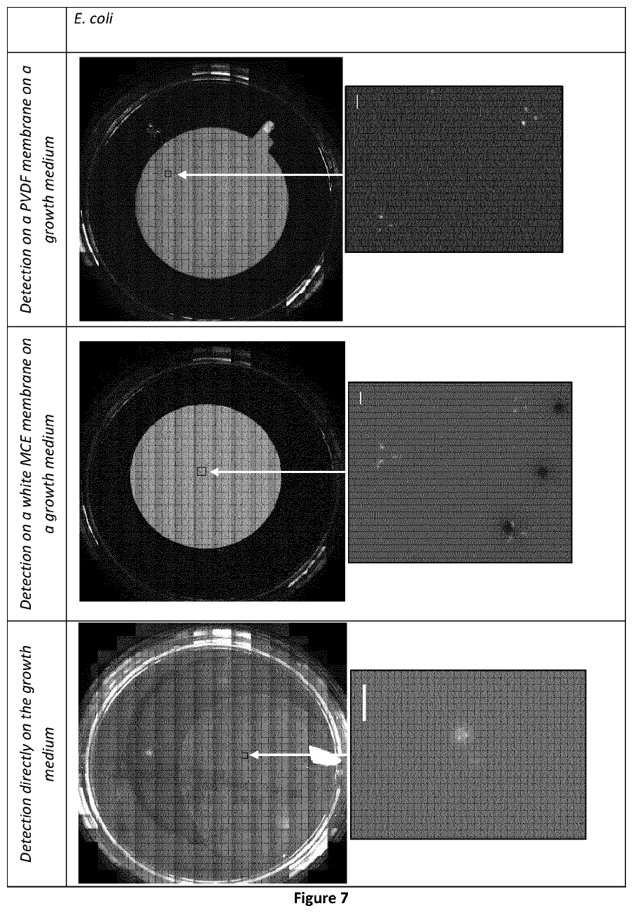

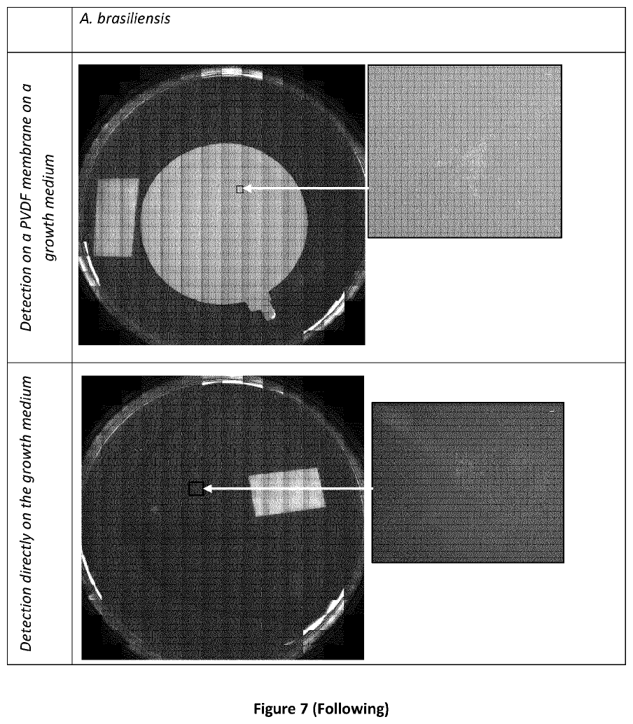

Examples of images acquired in each condition are presented in FIG. 7. These results show that detection of microorganisms can be conducted directly on the solid growth medium or on the membrane, even if said membrane is maintained on the growth medium.

### Using Different Set-Up Conditions to Minimize the Incubation Time Required to Detect Micro-Organisms

The method of the invention was investigated for different timings on C. albicans detection. Micro-organism was grown on white MCE membrane (either grided or ungrided) contacting SDA solid growth medium and growth was stopped at different timing (23 h, 25 h, or 28 h). For each timing investigated, the sample was imaged under different conditions: either standard conditions 0 (3 LEDs, incidence angle α set to 37°), either condition 1 (6 LEDs, incidence angle α set to 37°), either condition 2 (12 LEDs: 6 of them set to α=37°, 6 other set to α=50°). For each condition, the membrane was illuminated by collimated LEDs emitting white light, equidistant from each other and centred on the optical acquisition axis.

For each condition, the number of micro-organisms detected on the membrane after imaging was quantified (operator count, N) and the membrane was allowed to grow 48 hours again until colonies became visible by naked eye (CFU count). The percentage of detection was computed as the ratio N/CFU count.

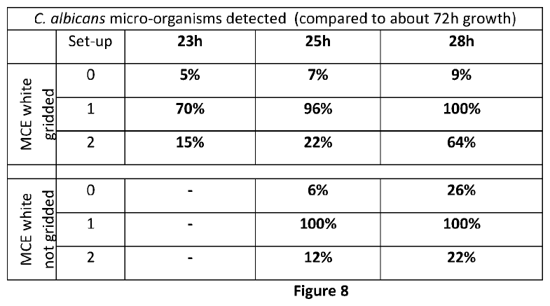

These percentages, obtained in each condition are presented in FIG. 8. These results show that some set-up conditions allow earlier detection of micro-organisms.

### Example 2

### Material and Methods

Detection of Microorganisms Implicated in Contamination of Raw Materials or Final Products from Pharmaceutical Industry and Cosmetics

Aspergillus brasiliensis ATCC® 16404™, Saccharomyces cerevisiae ATCC® 9763™, Zygosaccharomyces bailii DSM 70492, Penicillium chrysogenum ATCC® 18502,™ Candida albicans ATCC® 10231™ and Penicillium variotii ATCC® 18502™ were impacted on PVDF, black or white MCE filtration membrane depending on the strain. Membranes were deposited on SDA agar plates (Pancreatic Digest of Casein 5 g/L, Peptic Digest of Animal Tissue 5 g/L, Dextrose 40 g/L, Agar 15 g/L, pH value 5.6±0.2). Plates were then incubated between 24 h and 50 h (depending on the strain and the type of membrane) at 22.5° C.

Acinetobacter baumanii ATCC® 19606™, Aspergillus brasiliensis ATCC® 16404™, Bacillus subtilis ATCC® 6633™, Brevundimonas diminuta ATCC® 19146™, Burkholderia cepacia ATCC® 25608™, Candida albicans ATCC® 10231™, Clostridium sporogenes ATCC® 19404™, Cutibacterium acnes ATCC® 6919™, Enterobacter aerogenes ATCC® 35028™, Enterococcus faecalis ATCC™ 29212™, Escherichia coli ATCC® 8739™, Klebsiella pneumoniae ATCC® 13883™ Kocuria rhizophila ATCC® 9341™, Methylobacterium extorquens ATCC® 43645™, Proteus mirabilis ATCC® 29906™, Pseudomonas aeruginosa ATCC® 9027™, Ralstonia pickettii ATCC® 27511™, Serratia marcescens ATCC® 14756™, Shigella sonnei ATCC® 25931™, Staphylococcus aureus ATCC® 6538™, Streptococcus pneumoniae ATCC® 49619™ Vibrio parahaemolyticus ATCC® 17802™, Yersinia enterolitica ATCC® 9610™ were impacted on a PVDF, black or white MCE filtration membrane depending on the strain. Membranes were deposited on TSA agar plates (Tryptone 15 g/L, Soja papainic peptone 5 g/L, Sodium chloride 5 g/L, Agar 15 g/L, pH value 7.3±0.2). Plates were then incubated between 6 h and 72 h (depending on the strain and the type of membrane) at 32.5° C.

### Detection of Bacteria Implicated in Uncomplicated and Complicated UTI

E. coli ATCC® 8739™, K. pneumoniae ATCC® 13883™, P. mirabilis ATCC® 29906™, E. faecalis ATCC® 19433™ and S. aureus ATCC® 6538™ covering 89% of uncomplicated and complicated UTI were impacted on a PVDF and black MCE filtration membrane. Membranes were deposited on Columbia blood agar plates (Peptone 23 g/l, Starch 1 g/l, Sodium chloride 5 g/l, Sheep Blood 50 ml/I, Agar 14 g/l, pH value 7.3±0.2) for all strains and on Mac Conkey agar plates (peptone 20 g/L, lactose 10 g/L, Bile salts 1.5 g/L, crystal violet 0.001 g/L, neutral red 0.05 g/L, sodium chloride 5.0 g/L, Agar 15.0 g/L, pH value 7.1+/−0.2) for coliforms (E. coli and K. pneumoniae). Columbia agar plates were incubated between 7 h30 and 10 h45 (depending on the strain) at 37° C. Mac Conkey agar plates were incubated between 10 h and 11 h (depending on the strain and type of membrane) at 32.5° C.

### Detection of Microorganisms Implicated in Contamination of Main Waters and Waters for Pharmaceutical Use (Purified Waters and WFI Waters)

Gram negative bacteria used in this example were Acinetobacter baumanii ATCC® 19606™, Aeromonas hydrophila ATCC® 35654™, Brevundimonas diminuta ATCC® 19146™, Burkholderia cepacia ATCC® 25608™, Citrobacter freundii ATCC® 8090™, Edwardsiella tarda ATCC® 15947™, Enterobacter aerogenes ATCC® 35028™, Ochrobactrum anthropi CIP 82.115, Klebsiella pneumoniae ATCC® 13883™, Methylobacterium extorquens CIP 106787, Moraxella osloensis ATCC 19976®, Pantoea agglomerans ATCC® 27155™, Proteus mirabilis ATCC® 29906™, Pseudomonas aeruginosa ATCC® 10145™, Ralstonia pickettii ATCC® 27511™, Salmonella typhimurium ATCC® 13311™, Serratia marcescens ATCC® 13880™, Shigella sonnei ATCC® 25931™, Sphingomonas paucimobilis ATCC® 29837™, Stenotrophomonas maltophilia ATCC® 13637™, Vibrio parahaemolyticus ATCC® 17802™, Yersinia enterocolitica ATCC® 9610™ Gram positive bacteria are Bacillus subtilis ATCC® 6633™, Enterococcus faecalis ATCC® 19433™, Staphylococcus aureus ATCC® 6538™. Yeasts and molds tested were Aspergillus brasiliensis ATCC® 16404™ and Candida albicans ATCC® 10231™. This selection covers at least 84% of mains water systems microbial contaminants, 77% of purified water systems contaminants and the most prevalent contaminants of pharmaceutical WFI systems. Strains were impacted on a PVDF and black or white MCE filtration membrane (depending on the strain). Membranes were deposited on Reasoner's 2A Agar (R2A) plates (Yeast Extract 0.5 g/L, Proteose Peptone 0.5 g/L, Casein Hydrolysate 0.5 g/L, Glucose 0.5 g/L, Starch 0.5 g/L, Dipotassium Hydrogen Phosphate 0.3 g/L, Magnesium Sulphate, Anhydrous 0.024 g/L, Sodium Pyruvate 0.3 g/L, Agar 15.0 g/L, final pH 7.2±0.2). R2A plates were incubated between 24 h and 48 h (depending on the strain) at 32.5° C.

### Detection of Microorganisms Implicated in Spoilage or Contamination in Food Industry (Raw Materials and Final Products)

### General

Acinetobacter baumanii ATCC® 19606™, Bacillus subtilis ATCC® 6633™, Brevundimonas diminuta ATCC® 19146™, Enterobacter aerogenes ATCC® 35028™, Enterococcus faecalis ATCC™ 29212™, Kocuria rhizophila ATCC® 9341™, Shigella sonnei ATCC® 25931™, Streptococcus pneumoniae ATCC® 49619™, Vibrio parahaemolyticus ATCC® 17802™, Yersinia enterocolitica ATCC® 9610™ were impacted on PVDF filtration membranes. Membranes were deposited on PCA agar plates (Tryptone 5 g/L, Yeast extract 2.5 g/L, Dextrose 1 g/L, Agar 15 g/L, pH value 7.0±0.2). Plates were incubated between 18 h and 27 h (depending on the strain) at 35° C.

### Soft Drinks

Molds used in this example are Aspergillus brasiliensis ATCC® 16404™ and Penicillium variotii ATCC® 18502™. Acetic acid bacteria used are Acetobacter aceti ATCC® 15973™, Gluconoacetobacter liquefaciens ATCC® 14835™. Asaia siamensis DSM 15972 and Gluconobacter oxydans ATCC®193571™) and lactic acid bacterial strains used are Lactobacillus plantarum ATCC® 8014™, Weissella confusa ATCC® 10881™ and Lactobacillus casei ATCC® 393™. Strains were impacted on PVDF, black or white MCE filtration membrane (depending on the strain). Membranes were deposited on OSA (casein peptone 10 g/L, dipotassium hydrogen phosphate 3 g/L, D(+)-glucose 4 g/L, orange extract 5 g/L, yeast extract 3 g/L, agar 17 g/L, pH value 5.5±0.2), PDA (Potato Infusion 4 g/L, Dextrose 20 g/L, Agar 17 g/L, pH value 5.6±0.2) or YM agar plates (Glucose 10 g/L, Malt extract 3 g/L, Peptone 5 g/L, Yeast extract 3 g/L, Agar 15 g/L, pH value 6.2±0.2) for Aspergillus Brasiliensis and Penicillium variotii, on YM agar plates for acetic acid bacteria and on MRS agar plates (Diammonium hydrogen citrate 2 g/L, Dipotassium hydrogen phosphate 2 g/L, D(+)-glucose 20 g/L, Magnesium sulfate 0.1 g/L, Manganous sulfate 0.05 g/L, Meat extract 5 g/L, Sodium acetate 5 g/L, Universal peptone 10 g/L, Yeast extract 5 g/L, Agar 12 g/L, pH value 5.7) for lactic acid bacteria. OSA plates were incubated 40 h at 30° C. PDA plates were incubated 40 h at 25° C. YMA plates were incubated between 15 h and 72 h (depending on the strain and the type of membrane) at 25° C. or 30° C. MRS agar plates were incubated between 24 h and 26 h at 30° C.

### Fruit Juices

Strains of Alicyclobacillus bacteria including Alicyclobacillus acidoterrestris ATCC® 49025™, Alicyclobacillus acidocaldarius ATCC® 27009™, Alicyclobacillus acidiphilus DSM 14558, Alicyclobacillus cycloheptanicus ATCC® 49028™, Alicyclobacillus hesperidum DSM 12766, Alicyclobacillus herbarius DSM 13609 and Alicyclobacillus contaminans DSM 17975 were impacted on PVDF or black MCE filtration membrane (depending on the strain). Membranes were deposited on BAT agar plates (Yeast extract 2 g/l, D(+) glucose 5 g/l, Calcium chloride 0.25066 g/l, Magnesium sulfate 0.5 g/l, Ammonium sulfate 0.2 g/l, Potassium dihydrogen phosphate 3 g/l, Zinc sulfate 0.00018 g/l, Copper sulfate 0.00016 g/l, Manganese sulfate 0.00015 g/l, Sodium molybdate dihydrate 0,00030 g/l, Agar-Agar 18 g/l, pH value 3.8-4.2). BAT agar plates were incubated 24 h at 45° C.

### Wines

Saccharomyces cerevisiae (environmental strain), Zygosaccharomyces bailii (environmental strain), Lactobacillus nagelii (environmental strain) and Pediococcus pentosaceus ATCC® 33316™ were impacted on PVDF filtration membrane. Membranes were deposited on Tomato juice agar plates (Tomato juice 20 g/L, Peptone 10 g/L, Peptonised milk 10 g/L, Agar, 12 g/L, pH value 6.1±0.2). Tomato juice agar plates were incubated between 18 h and 72 h (depending on strain and cultural conditions) at 25° C. and 30° C.

### Sample Preparation

The membranes were deposited on a sample holder (1) made of black anodised aluminium along with 3 drops of sterile water (about 50-150 μl) to rehydrate and keep the membrane humidified. The cassette was then placed in position for imaging as illustrated in FIG. 1.

### Imaging System Setup

Membranes were imaged through a device as illustrated in FIG. 1 wherein the membrane (2) was placed on a sample holder (1) and was illuminated with 3 collimated LEDs (3) emitting white light, equidistant from each other and centred on the optical acquisition axis, and forming an angle (α) of 50° with respect to the normal of the support. An incident area of the membrane was imaged by means of a CMOS image sensor (4) having its optical acquisition axis along the normal of the membrane. The light reflected/scattered and/or diffused from the membrane and/or the microbiological objects on the membrane was detected.

Acquisition parameters (light source intensity 3 to 7 lumen/LED and camera exposure time 100 ms for microorganisms impacted on PVDF and black MCE membranes, light source intensity 1 to 5 lumen/LED and camera exposure time 40 to 50 ms for microorganisms impacted on MCE white membranes) were adjusted to maximize the signal-to-noise ratio. A multi-band emission filter (435±20 nm, 546±10 nm, and 690±50 nm bandpass) was placed between the CMOS image sensor and the sample.

### Results

TABLE 2
Detection of various microorganisms using the method of the invention

| | | | | | Detection | Detection |
|---|---|---|---|---|---|---|
| | | | | Detection | on MCE | on MCE |
| | | Growth | | on PVDF | black | white |
| Market | Microorganisms | medium | T° C. | membrane | membrane | membrane |
| Pharmaceutical | Aspergillus brasiliensis | SDA | 22.5° C. +/− | X | X | X |
| industry, | Saccharomyces cerevisiae | | 2.5° C. | X | / | X |
| cosmetics | Zygosaccharomyces bailii | | | X | / | X |
| | Penicillium chrysogenum | | | X | / | / |
| | Geotrichum candidum | | | X | / | / |
| | Candida albicans | | | X | X | X |
| | Penicillium variotii | | | X | / | / |
| | Acinetobacter baumanii | TSA | 32.5° C. +/− | X | / | / |
| | Aspergillus brasiliensis | | 2.5° C. | X | X | X |
| | Bacillus subtilis | | | X | X | X |
| | Brevundimonas diminuta | | | X | / | / |
| | Burkholderia cepacia | | | X | X | X |
| | Candida albicans | | | X | X | X |
| | Clostridium sporogenes | | | X | / | / |
| | Cutibacterium acnes | | | X | / | / |
| | Cutibacterium acnes | | | X | / | / |
| | Enterobacter aerogenes | | | X | / | / |
| | Enterococcus faecalis | | | X | X | / |
| | Escherichia coli | | | X | X | X |
| | Klebsiella pneumoniae | | | X | X | / |
| | Kocuria rhizophila | | | X | / | / |
| | (Micrococcus luteus) | | | | | |
| | Methylobacterium | | | X | X | X |
| | extorquens | | | | | |
| | Proteus mirabilis | | | X | / | / |
| | Pseudomonas aeruginosa | | | X | X | X |
| | Ralstonia picketii | | | X | X | / |
| | Serratia marcescens | | | X | / | / |
| | Shigella sonnei | | | X | / | / |
| | Staphylococcus aureus | | | X | X | X |
| | Streptococcus penumoniae | | | X | / | / |
| | Vibrio paraheamolyticus | | | X | / | / |
| | Yersinia enterolitica | | | X | / | / |
| Diagnosis (UTI) | Escherichia coli | Columbia | 37° C. | X | X | / |
| | Enterococcus faecalis | Blood | | X | X | / |
| | Klebsiella pneumonia | agar | | X | X | / |
| | Proteus mirabilis | | | X | X | / |
| | Staphylococcus aureus | | | X | X | / |
| | Escherichia coli | Mac | 32.5° C. | X | X | / |
| | Klebsiella pneumoniae | Conkey | | X | X | / |
| | | agar | | | | |
| Pharmaceutical | Acinetobacter baumanni | R2A | 32.5° C. | X | X | / |
| industry (main | Aeromonas hydrophila | | | X | X | / |
| water and | Aspergillus brasiliensis | | | X | X | X |
| waters for | Bacillus subtilis | | | X | X | X |
| pharmaceutical | Brevundimonas diminuta | | | X | X | X |
| use) | Burkholderia cepacia | | | X | X | X |
| | Candida albicans | | | X | X | X |
| | Citrobacter freundii | | | X | / | / |
| | Edwardiella tarda | | | X | / | / |
| | Enterobacter aerogenes | | | X | / | / |
| | Enterococcus faecalis | | | X | / | / |
| | Escherichia coli | | | X | X | X |
| | Klebsiella pneumoniae | | | X | / | / |
| | Methylobacterium | | | X | X | X |
| | extorquens | | | | | |
| | Moraxella osloensis | | | X | / | / |
| | Ochrobactrum anthropi | | | X | / | / |
| | Pseudomonas aeruginosa | | | X | X | X |
| | Pantoea agglomerans | | | X | / | / |
| | Proteus mirabilis | | | X | / | / |
| | Pseudomonas fluorescens | | | X | X | / |
| | Ralstonia picketii | | | X | X | / |
| | Salmonella typhimurium | | | X | / | / |
| | Serratia marcescens | | | X | / | / |
| | Shigella sonnei | | | X | / | / |
| | Sphingomonas | | | X | / | / |
| | paucimobilis | | | | | |
| | Stenotrophomonas | | | X | / | / |
| | maltophilia | | | | | |
| | Staphylococcus aureus | | | X | X | X |
| | Yersinia enterolitica | | | X | / | / |
| Food | Acinetobacter baumanii | PCA | 35° C. | X | / | / |
| | Kocuria | | | X | / | / |
| | Shigella sonnei | | | X | / | / |
| | Bacillus subtilis | | | X | / | / |
| | Enterococcus faecalis | | | X | / | / |
| | Brevundimonas diminuta | | | X | / | / |
| | Enterobacter aerogenes | | | X | / | / |
| | Vibrio paraheamolyticus | | | X | / | / |
| | Streptococcus penumoniae | | | X | / | / |
| | Yersinia enterolitica | | | X | / | / |
| Food (soft | Aspergillus brasiliensis | OSA | 30° C. | X | / | / |
| drinks) | Penicillium variotii | | | X | / | / |
| | Aspergillus brasiliensis | PDA | 25° C. | X | / | / |
| | Penicillium variotii | | | X | / | / |
| | Aspergillus brasiliensis | YMA | 25° C. | X | X | / |
| | Penicillium variotii | | | X | X | / |
| | Acinetobacter aceti | | 30° C. | X | X | X |
| | Asaia siamensis | | | X | / | X |
| | Gluconoacetobacter | | | X | X | X |
| | liquefaciens | | | | | |
| | Gluconobacter oxydans | | | X | / | X |
| | Lactobacillus casei | MRS | 30° C. | X | / | X |
| | Lactobacillus plantarum | agar | | X | / | / |
| | Weisella confusa | | | X | / | X |
| Food (fruit | Alicyclobacillus | BAT | 45° C. | X | / | / |
| juices) | acidoterrestris | agar | | | | |
| | Alicyclobacillus | | | X | / | / |
| | contaminans | | | | | |
| | Alicyclobacillus | | | X | / | / |
| | cycloheptanicus | | | | | |
| | Alicyclobacillus | | | X | / | / |
| | hesperidum | | | | | |
| | Alicyclobacillus herbarius | | | X | / | / |
| | Alicyclobacillus acidiphilus | | | X | / | / |
| | Alicyclobacillus | | | X | X | / |
| | acidocaldarius | | | | | |
| Food (wines) | Saccharomyces cerevisiae | Tomato | 25° C. | X | / | / |
| | Zygosaccharomyces bailii | Juice | | X | / | / |
| | Lactobacillus nagelii | Agar | | X | / | / |
| | Pediococcus pentosaceus | | | X | / | / |
| | Saccharomyces cerevisiae | | 30° C. | X | / | / |
| | Zygosaccharomyces bailii | | | X | / | / |
| | Lactobacillus nagelii | | | X | / | / |
| | Pediococcus pentosaceus | | | X | / | / |

(X) means that the microorganism of interest was detected.
(/) means that this condition was not tested.

### Example 3

### Material and Methods

### Preparation of Microorganisms

For A. brasiliensis, BioBalls® from Biomérieux containing a precise number of microorganisms (BioBall® Multishot 550 Aspergillus brasiliensis SKU number: 56001) were diluted in NaCl 0.9% to obtain a defined number of microorganisms per experimental condition impacted on a filtration membrane (density=100 objects/membrane). The membrane was deposited on SDA agar plates (Pancreatic Digest of Casein 5 g/L, Peptic Digest of Animal Tissue 5 g/L, Dextrose 40 g/L, Agar 15 g/L, pH value 5.6±0.2) and micro-organisms were allowed to grow for 38 h at 22.5° C.

Aspergillus brasiliensis (ATCC® 16404™), Candida albicans (ATCC® 10231™), Escherichia coli (ATCC® 8739™), Pseudomonas aeruginosa (ATCC® 9027™), Methylobacterium extorquens (NBRC 15911, ATCC® BAA-2500™), Ralstonia pickettii (ATCC® 27511™), Brevundimonas diminuta (ATCC® 19146™), Burkholderia cepacia (ATCC® 25416™), Staphylococcus aureus (ATCC® 6538™), Staphylococcus epidermidis (ATCC® 12228™), Pseudomonas fluorescens (ATCC® 17386™) and Bacillus subtilis (ATCC® 6633™) were diluted in NaCl 0.9% from cryopreserved stocks previously quantified to obtain a defined number of microorganisms per experimental condition (density=100 objects/membrane). The resulting suspensions were filtered on MCE white grided membranes (HAWG047, Merck Millipore) and micro-organisms were allowed to grow t hours at temperature T on solid growth media, i.e. on SDA, R2A or TSA agar plates. Conditions used to grow micro-organisms on filtration membranes are indicated in Table 3. For each sample, growth was stopped by removing the membrane from the solid growth medium and the sample was stored at 4° C. until imaging.

### Sample Preparation

The membranes were deposited on a sample holder (1) made of black anodised aluminium along with 3 drops of sterile water (about 50-250 μl) to rehydrate and keep the membrane humidified. The cassette was then placed in position for imaging as illustrated in FIG. 1.

### Imaging System Setup

Membranes were imaged through a device as illustrated in FIG. 1 wherein the membrane (2) was placed on a sample holder (1) and was illuminated with several incident collimated LEDs (3) emitting white light, each LED forming an angle (α) with respect to the normal of the support. An incident area of the membrane was imaged by means of a CMOS image sensor (4) having its optical acquisition axis along the normal of the membrane. The light reflected/scattered and/or diffused from the membrane and/or the microbiological objects on the membrane was detected.

The device used for the detection of Aspergillus brasiliensis comprised 6 light sources with a value of angle (α) of 370 evenly distributed on a circle centered on the optical acquisition axis.

The device used for the detection of Candida albicans and bacteria comprised 6 light sources with a value of angle (α) of 370 evenly distributed on a circle centered on the optical acquisition axis and 6 light sources with a value of angle (α) of 50° evenly distributed on another circle centered on the optical acquisition axis.

Acquisition parameters (light source intensities between 0.7 and 11 lumen/LED and camera exposure time between 100 and 300 ms) were adjusted to maximize the signal-to-noise ratio. A multi-band emission filter (435±20 nm, 546±10 nm, and 690±50 nm bandpass) was placed between the CMOS image sensor and the sample. The CMOS sensor used in the example was Sony IMX178LU-C (IDS imaging camera UI-3880CP-M-GL_R2 or UI-3880CP-C-GL_R2).

The percentage of detection was assessed by comparison to control condition, i.e. the number of colonies obtained after enough time to get visible colonies by eyes on the agar plate (with or without membrane).

### Results

Detection of Aspergillus brasiliensis

Two experimental setup were tested.

In the first setup, the membrane was illuminated simultaneously by 6 collimated LEDs equidistant from each other and centred on the optical acquisition axis. The incidence angle α was set to 37°. The image acquired in this condition was then used for detection of microorganisms. It was shown that this setup allowed the detection of 69% of Aspergillus brasiliensis microorganisms.

In the second setup, the membrane was illuminated sequentially by a first set of 3 collimated LEDs equidistant from each other and centred on the optical acquisition axis and by a second set of 3 collimated LEDs, distinct from the first set, equidistant from each other and centred on the optical acquisition axis. The incidence angle α was set to 37°. Images acquired with the first set and with the second set of 3 LEDs were then combined and used for detection of microorganisms. It was shown that this setup allowed the detection of 100% of Aspergillus brasiliensis microorganisms.

Detection of Candida albicans

Two experimental setup were tested.

In the first setup, the membrane was illuminated simultaneously by 6 collimated LEDs equidistant from each other, centred on the optical acquisition axis and having a angle α of 370 and 6 collimated LEDs equidistant from each other, centred on the optical acquisition axis and having a angle α of 50°. The image acquired in this condition was then used for detection of microorganisms. It was shown that this setup allowed the detection of 64% of Candida albicans microorganisms.

In the second setup, the membrane was illuminated sequentially by four sets of LEDs: a first set of 3 collimated LEDs having a angle α of 37°, a second set of 3 other collimated LEDs having a angle α of 37°, a third set of 3 collimated LEDs having a angle α of 50° and a fourth set of 3 other collimated LEDs having a angle α of 50°. Images acquired with each of these four sets were then combined and used for detection of microorganisms. It was shown that this setup allowed the detection of 100% of Candida albicans microorganisms.

### Detection of Bacteria

The second setup detailed above for the detection of Candida albicans was used to detect different bacteria. The results are presented in Table 3 below.

TABLE 3
Detection of various bacteria using the method of the invention

| | | | Incubation | % of |
|---|---|---|---|---|
| Strains | Media | Temperature | time | detection |
| E. coli ATCC 8739 | TSA | 32.5° C. | 8 hours | 92% |
| P. aeruginosa ATCC 9027 | TSA | 32.5° C. | 19 hours | 94% |
| M. extorquens ATCC 15911 | R2A | 22.5° C. | 72 hours | 100% |
| R. pickettii ATCC 27511 | R2A | 32.5° C. | 24 hours | 95% |
| B. diminuta ATCC 19146 | R2A | 32.5° C. | 42 hours | 100% |
| B. cepacia ATCC 25416 | TSA | 32.5° C. | 18 hours | 78% |
| S. aureus ATCC 6538 | TSA | 32.5° C. | 14 hours | 100% |
| S. epidermidis ATCC 12228 | TSA | 32.5° C. | 18 hours | 100% |
| P. fluorescens ATCC 17386 | R2A | 22.5° C. | 40 hours | 100% |
| B. subtilis ATCC 6634 | TSA | 32.5° C. | 16 hours | 100% |

1-51. (canceled)

52. A method for detecting microbiological objects on a support comprising
a) illuminating an area of the support with a set at least two incident collimated light sources forming an angle (α) of at least 10° with respect to the normal of the support, the value of angle (α) being independently selected for each incident collimated light source,
b) acquiring an image of said area of said support illuminated by said set of at least two incident collimated light sources by means of a light receiving element having its optical acquisition axis along the normal of the support, and
c) detecting the presence or the absence of microbiological objects on said area of said support by discrimination, on said acquired image, of light reflected, scattered and/or diffused from the support and from microbiological objects on said support.

53. The method according to claim 52, wherein the microbiological objects to be detected are selected from the group consisting of molds and colonies/microcolonies of bacteria, archaebacteria yeasts, and combinations thereof.

54. The method according to claim 52, wherein the microbiological objects to be detected are individualizable or individualized microbiological objects.

55. The method according to claim 52, wherein the microbiological objects to be detected are not labelled with a compound or moiety generating a photonic signal.

56. The method according to claim 52, wherein the support is a membrane filter, solid growth medium, or the support is in a container, optionally covered by a translucent lid.

57. The method according to claim 56, wherein the support is a membrane filter made of mixed cellulose esters (MCE), polyvinylidene fluoride (PVDF), polyester sulfone (PES), nitrocellulose, polytetrafluoroethylene, polycarbonate or nylon, or combinations thereof.

58. The method according to claim 52, wherein steps b) and c) are carried out a plurality of times to image several areas of said support or to image the entirety of the support.

59. The method according to claim 58, wherein the method further comprises a step d) of combining acquired images of said areas so as to form a combined image.

60. The method according to claim 52, wherein the method further comprises, after step b) and before step c),
b′) repeating steps a) and b) one or several times, each time with a different set of at least two incident collimated light sources forming an angle (α) of at least 10° with respect to the normal of the support, the value of angle (α) being independently selected for each incident collimated light source, thereby obtaining a plurality of acquired images of said area and combining said acquired images of said area so as to form a combined image of said area.

61. The method according to claim 60, wherein in step c) the presence or the absence of microbiological objects on said area of said support is detected by discrimination, on said combined image of said area, of light reflected, scattered and/or diffused from the support and from microbiological objects on said support.

62. The method according to claim 60, wherein steps a) and b) are repeated 1, 2, 3, 4, 5, 6, 7, 8, 9 or 10 times with 1, 2, 3, 4, 5, 6, 7, 8, 9 or 10 different sets of at least two incident collimated light sources, respectively.

63. The method according to claim 60, wherein:
the incident collimated light sources of two sets differ in their number, their type of light, their position and/or the value of angle α of each light sources; or
the incident collimated light sources of two sets differ in their number, their position and/or the value of angle α of each light sources.

64. The method according to claim 52, wherein steps a) and b) or steps a), b) and b′) are carried out a plurality of times to image several areas of said support or to image the entirety of the support.

65. The method according to claim 64, wherein the method further comprises after step b) or b′),
b″) combining acquired images of said areas so as to form a combined image of said areas.

66. The method according to claim 65, wherein in step c) the presence or the absence of microbiological objects on said areas of said support is detected by discrimination, on said combined image of said areas, of light reflected, scattered and/or diffused from the support and from microbiological objects on said support.

67. The method according to claim 52, wherein a set comprises: at least three incident collimated light sources; 3 to 24 incident collimated light sources; 3 to 12 incident collimated light sources; 3 to 6 incident collimated light sources; or 3 incident collimated light sources.

68. The method according to claim 52, wherein said incident collimated light sources of a set are evenly distributed on one or several circles centered on the optical acquisition axis.

69. The method according to claim 52, wherein each incident collimated light source forms an angle (α) which is independently selected between 15° to 75° with respect to the normal of the support, between 25° to 65° with respect to the normal of the support, or between 30° to 60° with respect to the normal of the support.

70. The method according to claim 52, wherein a set comprises at least three incident collimated light sources and the value of angle α is identical for all light sources of the set.

71. The method according to claim 52, wherein said incident collimated light sources are selected from collimated light-emitting diodes (LED) and laser diodes, and combination thereof.

72. The method according to claim 52, wherein the light receiving element is a camera comprising an array of pixel sensors, a CCD (charge-coupled device) image sensor or an active pixel sensor.

73. The method according to claim 52, further comprising enumerating and/or identifying microbiological objects on the support from acquired image(s) or combined image.

74. A device for detecting microbiological objects on a support comprising a support and at least two incident collimated light sources for illuminating the support, said at least two incident collimated light sources forming an angle (α) of at least 10° with respect to the normal of the support, the value of angle (α) being independently selected for each incident collimated light source,
a light receiving element for acquiring an image of an area of said support illuminated by said at least two incident collimated light sources, said light receiving element having its optical acquisition axis along the normal of the support, and
means detecting the presence or the absence of microbiological objects on said area of said support by discrimination, on said acquired image, of light reflected, scattered and/or diffused from the support and from microbiological objects on said support, and
optionally means to hold, and optionally move, the support.
